(19) United States
(12) Patent Application Publication
(10) Pub. No.: US 2015/0342165 A1
(43) Pub. Date: Dec. 3, 2015

(54) HUMANIZED MOUSE AND METHODS OF USING THE SAME

(71) Applicant: City of Hope, Duarte, CA (US)

(72) Inventors: David DiGiusto, Duarte, CA (US); Angel Sheng Rui Gu, Duarte, CA (US)

(21) Appl. No.: 14/722,326

(22) Filed: May 27, 2015

Related U.S. Application Data
(60) Provisional application No. 62/004,522, filed on May 29, 2014.

(51) Int. Cl. A01K 67/027; C12N 15/86; C12N 15/85

(52) U.S. Cl. CPC A01K 67/0278; C12N 15/8509; C12N 15/86; A01K 2207/15; A01K 2207/12; A01K 2227/105; C12N 2740/15043

(57) ABSTRACT

Provided herein, inter alia, is a humanized mouse with a gene-modified human hematopoietic stem and progenitor cell (GM-HSPC) graft, in which the HSPC cells within the graft are transduced with a gene vector, and gene vector includes a drug resistance gene or a disease treatment gene.

### RELATED APPLICATIONS

This application claims priority to, and the benefit of, U.S. provisional application No. 62/004,522, filed May 29, 2014, the disclosure of which is incorporated by reference in its entirety.

### BACKGROUND OF THE DISCLOSURE

Hematopoietic stem cell gene therapy is a promising therapeutic strategy for treating neoplastic, monogenic, and infectious disease. The expansion of adult hematopoietic stem and progenitor cells is of great interest in stem cell transplantation and gene therapy. The provision of increased numbers of hematopoietic stem and progenitor cells (HSPC) for autologous transplantation will improve rates of engraftment and increase the likelihood of successful replacement of defective or disease susceptible blood cells with functional and/or disease resistant versions.

The present disclosure relates to, in part, humanized mouse with a gene-modified human hematopoietic stem and progenitor cell (GM-HSPC) graft, in which the HSPC cells within the graft are transduced with a gene vector, and gene vector includes a drug resistance gene or a disease treatment gene.

### BRIEF SUMMARY OF THE DISCLOSURE

Provided herein, inter alia, is a humanized mouse with an adult human hematopoietic stem and progenitor cell (HSPC) graft. The present disclosure includes humanized mouse with a gene-modified human hematopoietic stem and progenitor cell (GM-HSPC) graft, in which the HSPC cells within the graft are transduced with a gene vector, and gene vector includes a drug resistance gene or a disease treatment gene.

In one aspect the HSPC graft is prepared by a method including contacting a plurality of isolated adult human HSPCs with cytokine culture media including stem cell factor (also known as SCF, KIT-ligand, KL, or steel factor), Flt3/flk-2 ligand (flt3-L) (a potent costimulator of normal bone marrow (BM) myeloid progenitors), thrombopoietin (Tpo), and interleulin-6 (IL-6), thereby forming a plurality of cultured-HSPCs. The plurality of cultured-HSPCs is allowed to undergo expansion, thereby forming a plurality of CD34+ HSPCs. At least about 5×10⁵ of the plurality of CD34+ HSPCs is administered to a mouse and the CD34+ HSPCs are allowed to engraft in the mouse to provide a HSPC-graft.

Also provided herein is humanized mouse with a gene-modified human hematopoietic stem and progenitor cell (GM-HSPC) graft formed by a method of this disclosure. The GM-HSPCs are generated by transducing adult HSPCs with a gene vector. The method includes contacting a plurality of isolated adult human CD34+ HSPCs with a media that includes SCF, Flt3-L, Tpo, and IL-6 to form a plurality of pre-stimulated HSPCs. The pre-stimulated HSPCs are contacted with a gene vector, to form a plurality of transduced-HSPCs (e.g., an HSPC having a stable transfected gene vector). The plurality of transduced-HSPCs is contacted with a cytokine media that includes SCF, Flt3-L, Tpo, and IL-6, which promotes growth and forms a plurality of GM-HSPCs. The GM-HSPCs are allowed to undergo expansion to form a plurality of GM-CD34+ HSPCs, thereby transducing the adult human HSPC with a gene vector. the GM-CD34+HSPCs are administered to a mousse and allowed to engraft in the mouse to provide a GM-HSPC-graft.

The methods described herein are useful for producing lymphoid cells from a subject in a humanized mouse. Such methods include, forming a HSPC-graft as described herein, including embodiments thereof, in a mouse and administering an effective amount of Fc/IL-7 to the mouse. The CD34+ HSPCs in the HSPC graft are then allowed to differentiate to lymphoid cells.

Likewise, the methods herein are useful for producing GM-lymphoid cells from a subject in a humanized mouse. Such methods include transducing isolated adult human HSPCs according to the methods described herein and administering the GM-HSPCs to a mouse to form a GM-HSPC as described herein. The mouse is administered an effective amount of Fc/IL-7 and the GM-CD34+ HSPCs in the graft are allowed to differentiate to GM-lymphoid cells.

The methods herein are also useful for producing GM-myeloid cells from a subject in a humanized mouse. Such methods include transducing isolated adult human HSPCs according to the methods described herein and administering the GM-HSPCs to a mouse to form a GM-HSPC as described herein. The GM-CD34+ HSPCs in the graft are allowed to differentiate to GM-myeloid cells.

Further provided herein is a humanized mouse. The humanized mouse includes a HSPC graft as described herein, including embodiments thereof. Alternatively, the humanized mouse includes a GM-HSPC graft as described herein, including embodiments thereof.

### BRIEF DESCRIPTION OF THE DRAWINGS

FIGS. 1A-1C depict graphs of in vitro Expansion of Adult HSPC: Phenotypic analysis of coordinated CD34 and CD90 expression before (FIG. 1A) and after (FIG. 1B) 7 days of in vitro expansion in SFT6+SR-1; (FIG. 1C) Fold expansion for TNC, CD34+, CD34+/CD90+ cells on day 7 (N=5 donors).

FIGS. 2A-2C depict graphs of engraftment of NSG mouse Bone marrow with minimally culture (MC) or expanded (Exp) HSPC: (FIG. 2A) The level of human cell engraftment at 16 weeks after transplant measured as a percentage of CD45+ cells in bone marrow based on CD34+MC-HSPC or Exp-HSPC dose, (FIG. 2B) CD34+/CD90+MC-HSPC or Exp-HSPC dose where the line represents regression curve for engraftment (N=174 mice for MC-HSPC and 110 mice for Exp-HSPC transplanted with cells form five healthy donors); (FIG. 2C) secondary engraftment of NSG mice with bone marrow isolated form MC-HSPC or Exp-HSPC engrafted primary mice; ***p<0.001.

FIGS. 3A-3D depict bar graphs of phenotypic distribution of CD45+ cells in the spleen and BM of humanized NSG mice: Average frequency (±SEM) of CD3+/CD4+ cell population in the spleen (FIG. 3A) and CD4+/CD14+, CD34+ and CD19+ cell populations in the bone marrow (FIGS. 3B-3D) are shown for different CD34+MC-HSPC and Exp-HSPC doses (M=million) (N=203 mice), *p<0.05, **p<0.01, ***p<0.001; NS=not significant; MC-HSPC=minimally cultured, Exp-HSPC=cultured for 7 days.

FIGS. 4A-4C depicts graphs of phenotypic analysis of human T-cells expanded from mice splenocytes: (FIG. 4A) Whole spleen-derived T-cells after REM1; (FIG. 4B) Whole spleen-derived T-cells after REM2; (FIG. 4C) CD45+/CD4+ Sorted cells after REM2. REM=rapid expansion method.

FIGS. 5A-5E depict graphs of engraftment of NSG mice in different organs: From mice peripheral blood (FIG. 5A) Bone Marrow (FIG. 5B) and Spleen (FIG. 5C) at 16 weeks post transplantation; Human CD45+ cells percentage from Bone Marrow (FIG. 5D), and Spleen (FIG. 5E) at 25 weeks post transplantation.

FIGS. 6A-6L depict fluorescent activated cell sorting (FACS) plots of phenotypic analysis of engrafted human blood cells. Plots show the level of human cell (CD45+) engraftment in bone marrow (FIGS. 6A-6C) and in spleen (FIGS. 6B-6I) 16 weeks after transplantation; and 25 weeks after transplantation in bone marrow (FIG. 6J) and in spleen (FIGS. 6K-6L). Bone marrow was stained simultaneously with antibodies to CD45, CD33, CD19, CD14 and CD34 (FIGS. 6A-6C). Splenocytes from the same mice were stained simultaneously with antibodies to CD45, CD3, CD4, CD8β, CD14 and CD19 (FIGS. 6D-6I). Similar staining of bone marrow (FIG. 6J) and spleen (FIGS. 6K-6L) at 25 weeks are shown for respective specimen obtained 25 weeks after transplantation.

FIG. 7 depicts a bar graph of frequency of T and B lymphoid and Monocytic lineages in the bone marrow and spleen of humanized NSG mice: Results from phenotypic analysis for lineage engraftment of >200 mice with an average frequency (±SEM) of each phenotypically defined population among the CD45+(human cells) is shown for each organ (Each mouse received 1×10⁶ CD34+ cells).

FIGS. 8A-8D depict graphs of administration of Fc-IL-7 fusion protein enhances human T-cell production in NSG mice: (FIG. 8A) FACS analysis of spleen from fresh cells engrafted mice without Fc-IL-7; gated on CD45+%; (FIG. 8B) FACS analysis of spleen from fresh cells engrafted mice with Fc-IL-7; gated on CD45+%; (FIG. 8C) HIV susceptible cells production in spleen from fresh cells engrafted mice; (FIG. 8D) HIV susceptible cells production in spleen from expanded cells engrafted mice (Mice were administrated with 20 μg Fc-IL-7 protein per mouse weekly and each mouse received 1×10⁶ CD34+ cells). (****p<0.0001, *p<0.05).

FIG. 9 depicts a graph of expansion of human CD3+ T-cells derived from mice splenocytes: (−) 153-fold expansion of T-cells at Day 14 after 2nd REM; (-•-) 91-fold expansion of T-cells in the CD45+/CD4+ sorted fraction culture. REM=rapid expansion method.

FIG. 10 depicts a graph of limiting dilution analysis of the frequency of NSG engrafting cells: The results from engraftment of NSG mice with limiting numbers of CD34+/CD90+ cells from MC-HSPC and Exp-HSPC is shown where the log fraction non-responding (non-engrafting) animals is shown on Y axis while CD34+/CD90+ cell dose is shown on X axis and MC-HSPC (N=68) Exp-HSPC (N=38) and downward triangles indicate the dose for each population at which there were no non-responding animals and dotted lines indicate 95% confidence interval for each population (MC-HSPC=minimally cultured. Exp-HSPC=cultured for 7 days).

### DETAILED DESCRIPTION OF THE DISCLOSURE

Provided herein, inter alia, is a humanized mouse and methods of making humanized mouse.

The following definitions are included for the purpose of understanding the present subject matter and for constructing the appended patent claims. Abbreviations used herein have their conventional meaning within the chemical and biological arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this disclosure. The following definitions are provided to facilitate understanding of certain terms used frequently herein and are not meant to limit the scope of the present disclosure.

The term “cell” as used herein also refers to individual cells, cell lines, or cultures derived from such cells. A “culture” refers to a composition comprising isolated cells of the same or a different type.

A “hematopoietic stem and progenitor cell” or “HSPC” as provided herein refers to a somatic stem cell that is able to give rise to (i.e. differentiate into) all blood cell lines. A hematopoietic stem cell is a subset of HSPC that has the capacity to differentiate into cells of the myeloid lineage (i.e. erythrocytes, mast cells, basophils, neutrophils, eosinophils, monocytes and macrophages) and the lymphoid lineage (i.e. T cells, B cells, and NK cells) and self-renew. A “peripheral blood HSPC” is a HSPC found in the circulating peripheral blood of a subject. A “cultured-HSPC” refers to a HSPC grown in cytokine culture media (e.g. a cell culture media including at least SCF, Flt3-L, Tpo, and IL-6). Cultured-HSPCs may be enriched (e.g. through mechanical isolation) via the expression of a particular protein or receptor (e.g. CD34). A “pre-stimulated HSPC” as used herein refers to a HSPC grown in a pre-stimulation media (e.g. a cell culture media including at least SCF, Flt3-L, Tpo, IL-6) prior to transduction with a gene vector as described herein. A “primitive HSPC” as used herein refers a HSPC which does not express the glycoproteins CD2, CD3, CD14, CD16, CD19, CD24, CD36, CD38, CD45RA, and CD56. A “GM-HSPC” as used herein, refers to a HSPC that is stably transfected with a gene vector as described herein, including embodiments thereof.

A “stem cell” is a cell characterized by the ability of self-renewal through mitotic cell division and the potential to differentiate into a particular cells, tissues or organs. As used herein, “multipotency” or “multipotent” or equivalents thereof refers to a cell type that can give rise to a limited number of other particular cell types. That is, multipotent cells are committed to one or more differentiated cell fates.

The term “derived from,” when referring to cells or a biological sample, indicates that the cell or sample was obtained from the stated source at some point in time. For example, a cell derived from an individual can represent a primary cell obtained directly from the individual (i.e., unmodified), or can be modified, e.g., by introduction of a recombinant vector, by culturing under particular conditions, or immortalization. In some cases, a cell derived from a given source will undergo cell division and/or differentiation such that the original cell is no longer exists, but the continuing cells will be understood to derive from the same source.

The terms “culture,” “culturing,” “grow,” “growing,” “maintain,” “maintaining,” “expand,” “expanding,” etc., when referring to cell can be used interchangeably to mean that the cell is maintained under conditions suitable for survival. Thus in embodiments, a cell may be expanded outside the body (e.g. ex vivo). In embodiments, cells may be expanded inside a subject (e.g. in vivo). In some instances, cells may be administered to a subject and allowed to expand within the subject to form a graft as described herein. Cells are allowed to survive, and can result in cell growth, differentiation, or division. The term does not imply that all cells in a cell culture survive or grow or divide, as some may naturally senesce, etc. Cells are typically cultured in media, which can be changed during the course of the culture. A cell may be part of a population of cells where the population may have the same genetic characteristics.

The terms “media” and “culture media” refer to the cell culture milieu. Media is typically an isotonic solution, and can be liquid, gelatinous, or semi-solid, e.g., to provide a matrix for cell adhesion or support. Media, as used herein, can include the components for nutritional, chemical, and structural support necessary for culturing a cell.

The terms “stem cell factor” and “SCF” are used interchangeably and according to their common, ordinary meaning and refer to cytokines of the same or similar names and functional fragments and homologs thereof. The term includes and recombinant or naturally occurring form of SCF (e.g. GI No: 1246100), or variants thereof that maintain SCF activity (e.g. within at least 30%, 40%, 50%, 60%, 70%, 80%, 90%, 95%, or 100% activity compared to SCF).

The term “Flt3-L” is used according to its common, ordinary meaning and refers to cytokines and ligands of the same or similar names and functional fragments and homologs thereof (e.g. “FMS-like tyrosine kinase-3 ligand” or “Flt3-LG”). The term includes and recombinant or naturally occurring form of Flt3-L (e.g. GI No: 219841754), or variants thereof that maintain Flt3-L activity (e.g. within at least 30%, 40%, 50%, 60%, 70%, 80%, 90%, 95%, or 100% activity compared to Flt3-L).

The terms “thrombopoietin” and “TPO” are used interchangeably and according to their common, ordinary meaning and refer to proteins of the same or similar names and functional fragments and homologs thereof. The term includes and recombinant or naturally occurring form of TPO (e.g. GI No: 914226), or variants thereof that maintain TPO activity (e.g. within at least 30%, 40%, 50%, 60%, 70%, 80%, 90%, 95%, or 100% activity compared to TPO).

The terms “interleukin 6”, “IL-6”, and “SFT6” are used interchangeably and according to their common, ordinary meaning and refer to cytokines of the same or similar names and functional fragments and homologs thereof. The term includes and recombinant or naturally occurring form of IL-6 (e.g., GI No: 4261586), or variants thereof that maintain IL-6 activity (e.g., within at least 30%, 40%, 50%, 60%, 70%, 80%, 90%, 95%, or 100% activity compared to IL-6).

The term “aryl hydrocarbon receptor antagonist” refers to a small molecule (e.g., a compound having a molecular weight less than about 1,000 Da), which enhances expansion or self-renewal of human HSPCs. In embodiments, an aryl hydrocarbon receptor antagonist increases expansion of CD34+ HSPCs. In embodiments, an aryl hydrocarbon receptor antagonist increases engraftment of a HSPC. In embodiments, the aryl hydrocarbon receptor antagonist is SR-1, CH-223191 or dimethyloxyflavone.

The terms “StemRegenin 1” and “SR-1” are used interchangeably and refer to a purine derivative having CAS. NO. 1227633-49-9 typically used to antagonize aryl hydrocarbon receptor signaling.

The term “leukocyte” is used herein according to its common, ordinary meaning and generally refers to all classes of white blood cells, including but not limited to, lymphocytes and monocytes. A “lymphocyte” or “lymphoid cell” is used interchangeably herein to refer to white blood cells bearing variable cell-surface receptors for antigen capable of mediating, for example, immunogenic responses. Lymphocyte as used herein includes all lymphocyte classes (e.g. T cells, B cells, natural killer cells) and subclasses therein which mediate humoral and/or cell mediated immunity. A “monocyte” refers to mononuclear white blood cells which are precursors to macrophages. A “natural killer cell” or “NK cell” refers to cytotoxic lymphocytes which can recognize stressed cells in the absence of antibodies and MHC.

The terms “differentiate,” “differentiation,” and “differentiating” are herein used interchangeably and refer to generation of a cell of a certain lineage (e.g., a lymphocyte such as a T-cell) from a different type of cell (e.g., a HSPC). A cell described herein which expresses a protein is indicated as (+) (e.g. a “CD34+ HSPC” refers to a HSPC which expresses the glycoprotein CD34). A cell which does not express a particular may be indicated as such (e.g. CD4−, CD8−, CD45RA−, CD62L−, CD25−, or CD127−). A “CD3+ T cell” as used herein refers to a T cell, which expresses the glycoprotein CD3. A “CD4+ T cell” as used herein refers to a T cell, which expresses the glycoprotein CD4. A “CD8+ T cell” as used herein refers to a T cell, which expresses the glycoprotein CD8. A “CD45RA+ T cell” as used herein refers to a T cell, which expresses the glycoprotein CD45RA. A “CD62L+ T cell” as used herein refers to a T cell, which expresses the glycoprotein CD62L. A “CD25+ T cell” as used herein refers to a T cell, which expresses the glycoprotein CD25. A “CD127+ T cell” as used herein refers to a T cell, which expresses the glycoprotein CD127. Thus, in embodiments, T-cells herein may express combinations of the aforementioned glycoproteins (e.g. CD3+/CD8+/CD4−; CD3+/CD4+/CD8−; or CD3+/CD4+ T-cells).

A “naïve” lymphocyte (e.g. naïve T-cell) refers to a lymphocyte that has not encountered antigen. A “memory” lymphocyte (e.g. T-cell) refers to a lymphocyte which mediates immunogenic response rapidly on re-exposure to an antigen that it previously encountered and for which it is specific. A “antigen specific” lymphocyte (e.g. T-cell) refers to a lymphocyte which is activated by its specific cognate antigen. A “tissue resident” lymphocyte (e.g. T-cell) refers to a lymphocyte present in a particular tissue (e.g. skin, airways, GI track) which provides cellular immunity.

As used herein, BCNU is a potent DNA alkylating agent that is used widely in cancer chemotherapy due to its cytotoxic effects. A cellular protein, methyl-guanine methyl-transferase (MGMT), repairs alkylating agent damage in cells but is sensitive to the inhibitory effects of O⁶-benzylguanine (O⁶BG). Treatment of cells with O⁶BG and BCNU results in the death of the cells.

The terms “graft,” “grafting,” “engraft” and the like are used interchangeably and according to their common, ordinary meaning and refer to transplantation of cells or tissues to a host from a genetically non-identical donor. The term may refer to a xenograft wherein a graft is formed via transplantation from a donor species (e.g. human) into a host species (e.g. mouse”). A “HSPC graft” refers to a graft formed from a plurality of HSPCs. A “GM-HSPC graft” refers to a graft formed from a plurality of GM-HSPCs as described herein. When cells or tissues from a human engraft into another host species (e.g. mouse), the host species is considered “humanized” (e.g. containing human cells which can grow, divide, expand, and/or differentiate within the host species).

“Patient,” “subject,” “patient in need thereof,” and “subject in need thereof” are herein used interchangeably and refer to a living organism suffering from or prone to a disease or condition that can be treated by administration using the methods and compositions provided herein. Non-limiting examples include humans, other mammals, bovines, rats, mice, dogs, monkeys, goat, sheep, cows, deer, and other non-mammalian animals. In some embodiments, a patient is human. Tissues, cells and their progeny of a biological entity obtained in vitro or cultured in vitro are also contemplated.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

By “co-administer” it is meant that a composition described herein is administered at the same time, just prior to, or just after the administration of one or more additional therapies. The compound of the disclosure can be administered alone or can be co-administered to the patient. Co-administration is meant to include simultaneous or sequential administration of the compound individually or in combination (more than one compound or agent). Thus, the preparations can also be combined, when desired, with other active substances (e.g. to reduce metabolic degradation).

The terms “treat,” “treating” or “treatment,” and other grammatical equivalents as used herein, include alleviating, abating or ameliorating a disease, condition or symptoms (e.g. hematological disease), preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder.

The terms “prevent,” “preventing,” or “prevention,” and other grammatical equivalents as used herein, include to keep from developing, occur, hinder or avert a disease or condition symptoms as well as to decrease the occurrence of symptoms. The prevention may be complete (i.e., no detectable symptoms) or partial, so that fewer symptoms are observed than would likely occur absent treatment. The terms further include a prophylactic benefit. For a disease or condition to be prevented, the compositions may be administered to a patient at risk of developing a particular disease (e.g. cancer or HIV), or to a patient reporting one or more of the physiological symptoms of a disease, even though a diagnosis of this disease may not have been made.

A “test compound” as used herein refers to an experimental compound used in a screening process to identify activity, non-activity, or other modulation of a particularized biological target or pathway.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

The term “predictive of a treatment outcome” refers to relating the outcome of treatment with a test compound in a cell or graft described herein, to the expected outcome of the subject who's cells were used to make the graft. Thus, in embodiments, when a test compound administered to a mouse having a HSPC-graft from a subject having a disease, and the compound treats the disease in the mouse, the compound may be expected to exhibit a similar or identical response when administered to the subject. Conversely, when a test compound administered to a mouse having a HSPC-graft from a subject having a disease, and the compound does not treat the disease in the mouse, the compound may be expected to exhibit a similar or identical response when administered to the subject.

“Disease” or “condition” refer to a state of being or health status of a patient or subject capable of being treated with the compounds or methods provided herein. In some instances, “disease” or “condition” refers to a “cancer” or “HIV” (i.e. Human Immunodeficiency Virus). As used herein, the term “cancer” refers to all types of cancer, neoplasm, malignant or benign tumors found in mammals, including leukemia, carcinomas and sarcomas. Exemplary cancers include breast cancer, ovarian cancer, colon cancer, liver cancer, kidney cancer and pancreatic cancer. Additional examples include leukemia (e.g. acute myeloid leukemia (“AML”) or chronic myelogenous leukemia (“CML”)), cancer of the brain, lung cancer, non-small cell lung cancer, melanoma, sarcomas, and prostate cancer, cervix cancers, stomach cancers, head & neck cancers, uterus cancers, mesothelioma, metastatic bone cancer, Medulloblastoma, Hodgkin's Disease, Non-Hodgkin's Lymphoma, multiple myeloma, neuroblastoma, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The murine leukemia model is widely accepted as being predictive of in vivo anti-leukemic activity. It is believed that a compound that tests positive in the P388 cell assay will generally exhibit some level of anti-leukemic activity regardless of the type of leukemia being treated. Accordingly, the present disclosure includes a method of treating leukemia, including treating acute myeloid leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas which can be treated with a combination of antineoplastic thiol-binding mitochondrial oxidant and an anticancer agent include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated with a combination of antineoplastic thiol-binding mitochondrial oxidant and an anticancer agent include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas which can be treated with a combination of antineoplastic thiol-binding mitochondrial oxidant and an anticancer agent include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, baso squamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “apheresis” refers to the process of separating the components (e.g. plasma (plasmapheresis), platelets (plateletpheresis) and leukocytes (leukapheresis)) of whole blood. Thus an “apheresis product” refers to the separated components of whole blood. In embodiments, an apheresis product refers to separated leukocytes from whole blood.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture. In some embodiments contacting includes allowing a compound described herein to interact with a protein or enzyme.

The terms “phenotype” and “phenotypic” as used herein refer to an organism's observable characteristics such as onset or progression of disease symptoms, biochemical properties, or physiological properties.

The word “expression” or “expressed” as used herein in reference to a DNA nucleic acid sequence (e.g. a gene) means the transcriptional and/or translational product of that sequence. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88). When used in reference to polypeptides, expression includes any step involved in the production of a polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The term “gene vector” refers to a nucleic acid molecule that encodes for genes and/or regulatory elements necessary for the expression of at least one gene. Expression of a gene from a gene vector can occur in cis- or in trans-. If a gene is expressed in cis-, gene and regulatory elements are encoded by the same plasmid. Expression in trans-refers to the instance where the gene and the regulatory elements are encoded by separate plasmids. Gene vectors contemplated herein include “drug resistance gene vectors” and “disease treatment gene vectors.” A drug resistance gene vector expresses at least one gene that conveys resistance of a protein or a cell to at least one drug (e.g. BCNU (e.g. bis-chloronitrosourea) or O⁶BG (O⁶-benzylguanine). A disease treatment gene vector expresses at least one gene having a gene product (e.g. polypeptide, polynucleotide, shRNA, snRNA, siRNA, snoRNA, or miRNA) useful for treating a disease. Gene vectors may include lentiviral gene vectors.

The term “exogenous” refers to a molecule or substance (e.g., a compound, nucleic acid (e.g. gene vector) or protein) that originates from outside a given cell or organism. Conversely, the term “endogenous” refers to a molecule or substance (e.g., a compound, nucleic acid (e.g. gene vector) or protein) native to, or originating within, a given cell or organism.

The terms “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “an amount of” in reference to a polynucleotide or polypeptide, refers to an amount at which a component or element is detected. The amount may be measured against a control, for example, wherein an increased level of a particular polynucleotide or polypeptide in relation to the control, demonstrates enrichment of the polynucleotide or polypeptide. Thus, in embodiments, an increased amount indicates a greater level or efficiency of grafting HSPCs described herein into a host (e.g. mouse). The term refers to quantitative measurement of the enrichment as well as qualitative measurement of an increase or decrease relative to a control.

The term “genetically modified-,” “genemodified-,” or “GM-” as used herein and appended to terms defined herein (e.g. HSPC, lymphoid cell), refers to stable incorporation of an exogenous gene or gene vector into the host such that the gene or gene vector continues to be expressed after multiple expansions.

Rapamycin is used herein according to is ordinary and common meaning and refers to an immunosuppressant drug commonly used to prevent rejection of tissue or organ transplantation. Rapamycin, as used herein, also includes known analogues and derivatives of rapamycin which retain rapamycin activity.

The terms “recapitulate” and “recapitulate a disease” as used herein refer to a cell (e.g. a HSPC or GM-HSPC) isolated from a subject and transplanted into a host, that is capable of regenerating or reintroducing the genotypic and/or phenotypic characteristics of the disease in the host. Thus, for example, a HSPC described herein isolated from a subject having HIV may regenerate or reintroduce a HIV genotype and/or phenotype into the host (e.g. a mouse).

### Mice

Provided herein is a humanized mouse. The humanized mouse includes a HSPC graft as described herein, including embodiments thereof. The HSPC graft may include CD34+HSPCs that differentiate into lymphoid or myeloid cells. In embodiments, the lymphoid or myeloid cells may be isolated from the mouse and optionally administered to a subject. Alternatively, the humanized mouse includes a GM-HSPC graft as described herein, including embodiments thereof. The GM-HSPC graft may include GM-CD34+ HSPCs that differentiate into GM-lymphoid or GM-myeloid cells. In embodiments, the GM-lymphoid or GM-myeloid cells may be isolated from the mouse and optionally administered to a subject.

### Methods for Grafting Adult Human HSPCs

Provided herein are methods useful for grafting adult human HSPCs. In embodiments, adult human HSPCs are grafted into a mouse. In one aspect, the method includes contacting a plurality of isolated adult human HSPCs with cytokine culture media including SCF, Flt3-L, Tpo, and IL-6, thereby forming a plurality of cultured-HSPCs. The plurality of cultured-HSPCs is allowed to undergo expansion, thereby forming a plurality of CD34+ HSPCs. At least 5×10⁵ of the plurality of CD34+ HSPCs is administered to a mouse and the CD34+ HSPCs are allowed to engraft in the mouse to provide a HSPC-graft. In embodiments, the isolated adult human HSPCs are administered to the mouse without contacting the HSPCs with the cytokine culture media or allowing the cells to under expansion. In embodiments, the method includes administering Fc/IL-7. The Fc/IL-7 may be administered at a therapeutic concentration such as those described herein. In embodiments, the plurality of isolated HSPCs is contacted with an aryl hydrocarbon receptor antagonist, as described herein. In embodiments, the method includes contacting the plurality of cultured-HSPCs with an aryl hydrocarbon receptor antagonist as described herein. In embodiments, the aryl hydrocarbon receptor antagonist is SR-1.

About 5×10⁵ to about 2×10⁶ CD34+ HSPCs may be administered. About 5×10⁵ to about 1.5×10⁶ CD34+ HSPCs may be administered. About 5×10⁵ to about 1×10⁶ CD34+HSPCs may be administered. In embodiments, at least 5×10⁵ to at least 2×10⁶ CD34+ HSPCs are administered. In embodiments, at least 1×10⁶ CD34+ HSPCs are administered. In embodiments, at least 2×10⁶ CD34+ HSPCs are administered.

In embodiments, the efficiency of the HSPCs grafting in said mouse may be measured. The efficiency may be a measure of the increase of the number of HSPCs that engraft in the mouse. The efficiency may be a measure of the enrichment of HSPCs expressing particular proteins in the plurality of CD34+ HSPCs. The efficiency may be measured by comparing amounts of HSPCs expressing certain CD glycoproteins (e.g. CD34, CD90). Thus, in embodiments, the method includes determining an amount of CD34+ HSPCs within the HSPC graft, to provide a post-engrafting CD34+ HSPC amount. The post-engrafting CD34+ amount may then be compared to the amount of plurality of CD34+ HSPCs administered to the mouse. In embodiments, the fold-increase of the CD34+ HSPCs post-engrafting is about 1.1 to about 20-fold increased relative to the amount of the CD34+ HSPCs prior to administration. The fold-increase may be about 1.1 to about 10-fold increased. The fold-increase may be about 1.1 to about 5-fold increased. The fold-increase may be at least 1.1 to at least 20-fold increased. The fold-increase may be about 1.1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, or 20-fold. The fold-increase may be about 5, 10, 15, or 20-fold. The fold-increase may be at least about 1.1, 1.5, 2, 2.5, 3, 3.5, 4, 4.5, or 5-fold. The fold increase may be at least about 1.1-fold.

In embodiments, the HSPC-graft includes primitive HSPCs. The method may include determining a population of primitive HSPCs within the plurality of CD34+ HSPCs before administering the plurality of CD34+ HSPCs to the mouse to provide a pre-engrafting primitive HSPC level. The method may include determining a population of primitive HSPCs within the HSPC graft to provide a post-engrafting primitive HSPCs level. Accordingly, the level of primitive HSPCs in the HSPC-graft and/or the enrichment of primitive HSPCs in the HSPC-graft may be determined by comparing the post-engrafting primitive HSPCs level to the pre-engrafting primitive HSPCs level. Thus, in embodiments, the methods include enriching the expression of CD glycoproteins (e.g. CD34, CD90). In embodiments, the fold-increase of the primitive HSPCs post-engrafting is about 1.1 to about 10-fold increased relative to the amount of the primitive HSPCs prior to administration. The fold-increase may be at least 1.1 to at least 5 fold increased. The fold-increase may be at least 1.1 to at least 2 fold increased. The fold-increase may be at least 1.1 fold. The fold-increase may be at least 2 fold.

In embodiments, the plurality of CD34+ HSPCs differentiate into a myeloid cell or a lymphoid cell as described herein. In such instances, the plurality of CD34+ HSPCs may differentiate into cells of myeloid lineage such as a monocyte or a macrophage. Alternatively, plurality of CD34+ HSPCs may differentiate into cells of lymphoid lineage such as a T cell, B cell, or NK cell. The plurality of CD34+ HSPCs may differentiate into myeloid or lymphoid cells characterized by immunophenotyping the cell (i.e. determining the varying expression of the cell's receptors and or ligands such as CD (“cluster of differentiation” molecules)). Thus, CD34+ HSPCs differentiating into lymphoid lineages may differentiate to T-cells, B-cells, and NK cells which independently exhibit different immunophenotypes. In embodiments, the CD34+ HSPCs described herein differentiate to lymphoid cells. The lymphoid cells may be CD3+/CD8+/CD4− T-cells, CD3+/CD4+/CD8− T-cells, or CD3+/CD4+ T-cells. The lymphoid cells may be CD3+/CD8+/CD4− T-cells. The lymphoid cells may be CD3+/CD4+/CD8− T-cells. The lymphoid cells may be CD3+/CD4+ T-cells. The lymphoid cells may be CD16+/CD56+NK cells. The lymphoid cells may be CD19+ B-cells. The lymphoid cells may be CD4+/CD14+ monocytes. In embodiments, the lymphoid cells are CD19+ B-cells, CD4+/CD14+ monocytes, or CD16+/CD56+ NK cells.

The lymphoid cells described herein may express CD45RA, CD62L, CD25, or CD127. Thus, in embodiments, the lymphoid cells described herein are CD45RA+ T-cells or CD62L+ T-cells. In embodiments, the lymphoid cells described herein are at least one of CD45RA+ T-cells or CD62L+ T-cells. The lymphoid cells may be CD45RA+ T-cells. The lymphoid cells may be CD62L+ T-cells. In embodiments, the lymphoid cells described herein are CD25+ T-cells or CD127+ T-cells. In embodiments, the lymphoid cells described herein are at least one of CD25+ T-cells or CD127+ T-cells. The lymphoid cells may be CD25+ T-cells. The lymphoid cells may be CD127+ T-cells. The lymphoid cells may be CD45+ T-cells. The lymphoid cells may be CD90+ T-cells. Lymphoid cells herein may be cumulative in their expression of glycoproteins (e.g. a lymphoid cell may express at least two different CD molecules (e.g. a CD3+/CD4+ T-cell may also be CD45RA+ and/or CD25+).

When the lymphoid cells are T-cells, the T-cells may be naïve, memory, antigen specific, or tissue resident T-cells. The T-cells may be naïve T-cells. The T-cells may be memory T-cells. The T-cells may be antigen specific T-cells. The T-cells may be tissue resident T-cells. Each naïve, memory, antigen specific, or tissue resident T-cell described herein may have a varied immunophenotype (e.g. expression of receptors or ligands such as CD molecules) as described herein.

The plurality of CD34+ HSPCs may be CD90+ HSPCs. The plurality of CD34+HSPCs may be CD49f HSPCs. The plurality of CD34+ HSPCs may be CD45+ HSPCs. In embodiments, the CD molecule expression of the plurality of CD34+ HSPCs described herein, including embodiments thereof, may be cumulative (e.g. a CD34+ HSPC may also be at least one of CD90+, CD49f+ or CD45+).

Also provided herein are methods of transducing an adult hematopoietic stem cell (HSPC). In one aspect, the method includes contacting a plurality of isolated adult human CD34+ HSPCs with a pre-stimulation media that includes SCF, Flt3-L, Tpo, and IL-6 to form a plurality of pre-stimulated HSPCs. The pre-stimulated HSPCs are contacted with a gene vector, to form a plurality of transduced-HSPCs (e.g. an HSPC having a stable transfected gene vector). The plurality of transduced-HSPCs is contacted with a growth promoting cytokine media that includes SCF, Flt3-L, Tpo, and IL-6, to form a plurality of GM-HSPCs. The GM-HSPCs are allowed to undergo expansion to form a plurality of GM-CD34+ HSPCs, thereby transducing the adult human HSPC with a gene vector. In embodiments, the plurality of isolated human CD34+HSPCs is contacted with an aryl hydrocarbon receptor antagonist. In embodiments, the plurality of pre-stimulated HSPCs is contacted with an aryl hydrocarbon receptor antagonist. In embodiments, the plurality of transduced-HSPCs is contacted with an aryl hydrocarbon receptor antagonist. In embodiments, the aryl hydrocarbon receptor antagonist is SR-1.

The adult human CD34+ HSPCs described herein, including embodiments thereof, may be isolated using standard apheresis and selection techniques known in the art. In embodiments, the plurality of pre-stimulated HSPCs is contacted with a gene vector in the presence of rapamycin.

In embodiments, the method includes grafting the GM-HSPCs into a mouse. The mouse may be a NSG (i.e., NOD scid IL2 receptor gamma chain knockout) mouse. The NSG mouse may be irradiated prior to administering the GM-HSPCs. At least 5×10⁵ GM-CD34+HSPCs may be administered to the mouse. The GM-CD34+ HSPCs administered to the mouse may be allowed to engraft in the mouse to form a GM-HSPC graft.

In embodiments, the plurality of GM-CD34+ HSPCs differentiate into a GM-myeloid cell or a GM-lymphoid cell as described herein. In such instances, the plurality of GM-CD34+HSPCs may differentiate into cells of myeloid lineage such as a GM-monocyte or a GM-macrophage. Alternatively, the plurality of GM-CD34+ HSPCs may differentiate into cells of lymphoid lineage such as a GM-T cell, a GM-B cell, or a GM-NK cell. The plurality of GM-CD34+ HSPCs may differentiate into myeloid or lymphoid cells characterized by immunophenotyping the cell (i.e. determining the varying expression of the cell's receptors and or ligands such as CD (“cluster of differentiation” molecules)) as described herein for CD34+HSPCs.

The GM-CD34+ HSPCs of the graft may differentiate into GM-lymphoid cells. The GM-lymphoid cells include at least one gene vector. The GM-lymphoid cells may be the type of lymphoid cells described hereinabove for lymphoid cells, including embodiments thereof. Thus, in embodiments, the GM-lymphoid cells include at least one gene vector (e.g. a GM-CD3+/CD8+/CD4− T-cell, GM-CD3+/CD4+/CD8− T-cell, GM-CD3+/CD4+ T-cell, GM-CD19+ B-cell, GM-CD4+/CD14+ monocyte, GM-CD16+/CD56+ NK cell). The GM-lymphoid cell may be a GM-T-cell, a GM-B-cell, or a GM-NK cell. In embodiments, the plurality of GM-CD34+ HSPCs differentiates into a GM-myeloid cell.

The gene vector may be plasmid (e.g. a nucleic acid molecule that encodes for genes and/or regulatory elements necessary for the expression of genes) or a viral vector. The gene vector may be a retroviral gene vector such as a lentiviral gene vector. In embodiments, the gene vector includes at least one of a drug resistance gene or a disease treatment gene. The gene vector may include a drug resistance gene or a disease treatment gene. The gene vector may include a drug resistance gene and a disease treatment gene. The gene vector may include a drug resistance gene. The drug resistance gene may be a BCNU-resistant drug resistance gene. The drug resistance gene may be a O⁶BG-resistant drug resistance gene.

The gene vector may include a disease treatment gene. The disease treatment gene may be a gene that encodes for a polypeptide, protein, antibody, polynucleotide, shRNA, snRNA, siRNA, snoRNA, or miRNA. The polypeptide, protein, antibody, polynucleotide, shRNA, snRNA, siRNA, snoRNA, or miRNA may be useful in treating a disease. In embodiments, the disease treatment gene is a HIV disease treatment gene which expresses a polypeptide, protein, antibody, polynucleotide, shRNA, snRNA, siRNA, snoRNA, or miRNA which treats HIV. In embodiments, the HIV disease treatment gene is a composition as described by PCT publication WO 15/042308, which is fully incorporated herein by reference. In embodiments, the disease treatment gene is a cancer disease treatment gene (i.e. a gene useful for treating one or more symptoms associated with cancer). The cancer may be breast cancer, ovarian cancer, colon cancer, liver cancer, kidney cancer, pancreatic cancer, leukemia, brain cancer, lung cancer, non-small cell lung cancer, melanoma, sarcoma, prostate cancer, cervix cancer, stomach cancer, testicular cancer, lymphoma, or thyroid cancer. The cancer may be breast cancer or ovarian cancer. The cancer may be colon cancer, liver cancer, kidney cancer or pancreatic cancer. The cancer may be leukemia. The cancer may be lung cancer or non-small cell lung cancer. The cancer may be melanoma. The cancer may be sarcoma. The cancer may be prostate cancer.

In embodiments, the gene vector is administered at a multiplicity of infection (i.e. ratio of gene vector to HSPCs) of at least 5 to at least 100. The gene vector may be administered at a multiplicity of infection of at least 10 to at least 100. The gene vector may be administered at a multiplicity of infection of at least 10 to at least 90. The gene vector may be administered at a multiplicity of infection of at least 10 to at least 80. The gene vector may be administered at a multiplicity of infection of at least 10 to at least 70. The gene vector may be administered at a multiplicity of infection of at least 10 to at least 60. The gene vector may be administered at a multiplicity of infection of at least 10 to at least 50. The gene vector may be administered at a multiplicity of infection of at least 10 to at least 40. The gene vector may be administered at a multiplicity of infection of at least 10 to at least 30. The gene vector may be administered at a multiplicity of infection of at least 10 to at least 20. The gene vector may be administered at a multiplicity of infection of at least 10. The gene vector may be administered at a multiplicity of infection of at least 5. The gene vector may be administered at a multiplicity of infection of at least 20. The gene vector may be administered at a multiplicity of infection of at least 30. The gene vector may be administered at a multiplicity of infection of at least 40. The gene vector may be administered at a multiplicity of infection of at least 50. The gene vector may be administered at a multiplicity of infection of at least 60. The gene vector may be administered at a multiplicity of infection of at least 70. The gene vector may be administered at a multiplicity of infection of at least 80. The gene vector may be administered at a multiplicity of infection of at least 90. The gene vector may be administered at a multiplicity of infection of at least 100.

In embodiments, the plurality GM-CD34+ HSPCs is administered to a subject. In embodiments, the plurality GM-CD34+ HSPCs is administered to a subject having a disease. The disease may be treated by administering the plurality GM-CD34+ HSPCs to the subject. In embodiments, the disease treated is cancer or HIV. The cancer is as described herein, including embodiments thereof.

The methods described herein are useful for producing lymphoid cells from a subject in a humanized mouse. Such methods include, forming a HSPC-graft as described herein, including embodiments thereof, in a mouse (e.g. NSG mouse) and administering an effective amount of Fc/IL-7 to the mouse. The CD34+ HSPCs in the HSPC graft are then allowed to differentiate to lymphoid cells. In embodiments, the lymphoid cells may be isolated from the mouse using techniques known in the art. The lymphoid cells are as described herein, including embodiments thereof. The lymphoid cells may be administered to a subject to treat a disease as described herein.

Likewise, the methods herein are useful for producing GM-lymphoid cells from a subject in a humanized mouse. Such methods include transducing isolated adult human HSPCs according to the methods described herein and administering the GM-HSPCs to a mouse (e.g. NSG mouse) to form a GM-HSPC as described herein. The mouse is administered an effective amount of Fc/IL-7 and the GM-CD34+ HSPCs in the graft differentiate to GM-lymphoid cells. The Fc/IL-7 may be administered as a single dose or multiple doses. The Fc/IL-7 may be administered once a day for an extended period of time. In embodiments, the Fc/IL-7 may be administered for about 1, 2, 3, 4, 5, 6, 7, 8, 9, 10, 15, 20, 25, 30, 35, 40, 45, 50, 75, or 100 days. The Fc/IL-7 may be administered for about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks.

The Fc/IL-7 may be administered for about 1 to about 30 weeks. The Fc/IL-7 may be administered for about 1 to about 25 weeks. The Fc/IL-7 may be administered for about 1 to about 20 weeks. The Fc/IL-7 may be administered for about 1 to about 15 weeks. The Fc/IL-7 may be administered for about 1 to about 10 weeks. The Fc/IL-7 may be administered for about 1 week. The Fc/IL-7 may be administered for about 2 weeks. The Fc/IL-7 may be administered for about 5 weeks. The Fc/IL-7 may be administered for about 10 weeks. The Fc/IL-7 may be administered for about 15 weeks. The Fc/IL-7 may be administered for about 20 weeks. The Fc/IL-7 may be administered for about 25 weeks. In embodiments, the GM-lymphoid cells may be isolated from the mouse using techniques known in the art. The GM-lymphoid cells are as described herein, including embodiments thereof. The GM-lymphoid cells may be administered to a subject to treat a disease as described herein.

The methods herein are useful for producing GM-myeloid cells from a subject in a humanized mouse. Such methods include transducing isolated adult human HSPCs according to the methods described herein and administering the GM-HSPCs to a mouse (e.g. NSG mouse) to form a GM-HSPC as described herein. The GM-CD34+ HSPCs in the graft differentiate to GM-myeloid cells. In embodiments, the mouse is administered an effective amount of Fc/IL-7 as described herein.

In embodiments, a portion of the isolated adult human HSPCs may first be transduced according to the methods described herein to form transduced-HSPCs. In such embodiments, the transduced-HSPCs may be expanded and injected into a mouse according to the methods set forth herein, and in certain embodiments, allowed to differentiate into GM-lymphoid cells or GM-myeloid cells. In embodiments, GM-lymphoid cells described herein may be injected into a mouse (e.g. a mouse that recapitulates a disease state of a subject described herein). In embodiments, GM-myeloid cells described herein may be injected into a mouse (e.g. a mouse that recapitulates a disease state of a subject described herein). The GM-lymphoid or GM-myeloid cells may treat the disease. Thus, in embodiments, the GM-lymphoid cells may change either the phenotype or genotype of the mouse as described herein. In embodiments, the GM-myeloid cells may change either the phenotype or genotype of the mouse as described herein.

In embodiments, the isolated adult HSPCs are from a subject having a disease. The disease may be cancer or HIV as described herein. The HSPCs may be adult peripheral blood HSPCs. The HSPCs may be obtained from apheresis product as described herein. The HSPCs may be expanded as described herein and injected into a mouse using the methods set forth herein. The HSPCs may be allowed to differentiate into lymphoid cells as described herein. In embodiments, the lymphoid cells recapitulate the disease of the subject if injected into a mouse. Thus, in embodiments, the mouse having a HSPC-graft as described herein, exhibits at least one of the identical phenotype or the identical genotype of the individual having the disease.

The method may further include identifying a test compound which treats the disease by administering a test compound to the mouse having a HSPC-graft. The test compound can be analyzed to determine if it treats the disease by detecting a change in either the phenotype or genotype of the mouse. A change in the phenotype may indicate that the test compound treats the disease. The change in phenotype may be amelioration or prevention of the symptoms of the disease. Conversely, a change in the phenotype, or no change in the phenotype may indicate that the test compound does not treat the disease. The change in phenotype may indicate toxicity or unwanted interactions caused by the test compound. Thus, in embodiments, the mouse having HSPC-graft graft is a model for determining the treatment strategy of a disease.

In embodiments, the phenotypic or genotypic change is predictive of a treatment outcome for the subject. Thus, administration of a test compound may demonstrate treatment of the disease in the mouse described herein and may indicate that the compound may be effective for treatment of the subject. Thus, in embodiments, the outcome of the treatment in the mouse is indicative of the outcome of the treatment in the subject. In other embodiments, GM-lymphoid cells in the mouse may provide therapeutic treatment of the disease recapitulated by the CD34+HSPCs administered to the mouse. Thus, the GM-lymphoid cells may ameliorate or prevent the symptoms of the disease, such as HIV. In embodiments, the lymphoid cells or GM-lymphoid cells are administered (e.g. transplanted) to the subject. Administration of the lymphoid cells or GM-lymphoid cells may treat the disease in the subject.

In embodiments, GM-myeloid cells, as described herein, in the mouse may provide therapeutic treatment of the disease recapitulated by the GM-CD34+ HSPCs administered to the mouse. Thus, the GM-myeloid cells may ameliorate or prevent the symptoms of the disease, such as HIV. In embodiments, the myeloid cells or GM-myeloid cells are administered (e.g. transplanted) to the subject. Administration of the myeloid cells or GM-myeloid cells may treat the disease in the subject.

### EXAMPLES

### Example 1

### Bone Marrow of Animals Transplanted with Either Fresh or Expanded Adult HSPC Contained Secondary Engrafting Cells at 16 Weeks

### Mouse Models

NOD.Cg-Prkdc^{scid} IL2^{rgtm1}Wjl/SzJ (NSG) mice were obtained from The Jackson Laboratory (Bar Harbor, Me.) and bred at the City of Hope Animal Resources Center.

### In Vivo O⁶BG and BCNU Selection:

All animals except control mice received 20 mg/kg O⁶BG (Sigma-Aldrich, St. Louis, Mo.) followed by 5 mg/kg BCNU (Sigma-Aldrich, St. Louis, Mo.) mouse 1.5 hour later via IP injection. Drug doses were administered at either at the 7th and 8th or 7th, 8th, and 9th weeks post-transplantation. O⁶BG was prepared in 35% PEG-400 (Sigma-Aldrich, St. Louis, Mo.) and 60% injectable saline.

### Cells

Human mobilized hematopoietic progenitor cells were obtained by Progenitor Cell Therapies (Allendale, N.J.) and Key Biologics (Memphis, Tenn.) from healthy donors under informed consent. CD34+ HSPC were isolated as previously described in Tran CA et al. Optimized processing of growth factor mobilized peripheral blood CD34+ products by counterflow centrifugal elutriation. Stem cells translational medicine. 2012; 1:422-429. CD34-enriched HSPC cells were evaluated by flow cytometry for the coordinated expression of CD34, CD90 and CD49f to determine the distribution of progenitors and more primitive stem cells as previously described in Notta F, et al. Isolation of single human hematopoietic stem cells capable of long-term multi-lineage engraftment. Science. 2011; 333:218-221. CD34-enriched HSPC were pre-stimulated overnight in media (StemSpan, Stem Cell Technologies, Vancouver, Canada) containing 100 ng/ml each of stem cell factor (SCF), FMS-like tyrosine kinase-3 ligand (Flt-3L) (Invitrogen, Carlsbad, Calif.), 10 ng/ml of thrombopoietin (TPO) (Cell Genix GmbH, Freiburg, Germany) and 50 ng/ml of interleukin-6 (IL-6) hereinafter referred to as SFT6. HSPC were also cultured for up to 7 days in SFT6 plus 0.75 μM SR-1 (Cellagen Technology, San Diego, Calif.) to promote the expansion of HSPC.

### Cell Sorting

Splenocytes from engrafted mice were stained with CD45-PC7 and CD4-PC5 antibodies (BioLegend, San Diego, Calif.) as described above in PBS plus 90 U/ml Benzonase, 10 U/mL heparin and 0.1% bovine serum albumin (all from Sigma-Aldrich, St. Louis, Mo.). Cells were washed 3 times and resuspended into approximately 10 ml of 10% FBS enriched Benzonase-buffer before loading them into an Owl Nanosorter (Owl Biomedical, Santa Barbara, Calif.) cartridge. Cells expressing both CD45 and CD4 were sorted in enrich mode as per manufacturer's directions. Cells from each sort were analyzed for purity using a Gallios flow cytometer.

### T-Cell Culture

Splenocytes or CD45+/CD4+ sorted T-cells were expanded in vitro using the Rapid Expansion Method (REM) modified as previously described in Tran CA, et al. Manufacturing of Large Numbers of Patient-specific T Cells for Adoptive Immunotherapy: An Approach to Improving Product Safety, Composition, and Production Capacity. J Immunother (1997). 2007; 30:644-654. Briefly, splenocytes were cultured in RPMI 1640 with 10% FBS, 2 mM L-Glutamine, 25 mM HEPES, 50 IU/ml rIL-2 (Prometheus Therapeutics & Diagnostics, San Diego, Calif.), 10 ng/ml IL-15 (CellGenix, Freiburg, Germany) and 30 ng/ml anti-CDR antibody (Miltenyi Biotec, Inc., Auburn, Calif.) for 14 days. For CD45+/CD4+ sorted T-cells (and T-cells derived from splenocytes) cultures were initiated with 2-7×10⁴ T-cells and irradiated allogeneic PBMC at 2.4×10⁶ cells/mL in RPMI+FBS and cytokines as described above. Cells were stained with antibodies to CD45-PC7 (BioLegend, San Diego, Calif.), CD3-APC-Alexa750, CD4-PE-Texas Red (Invitrogen Carlsbad, Calif.), CD62L-PE, CD45RA-APC, CD25-PE, CD137-PE, CD8β-APC, CD28-APC, T-cell receptor γδ-PE (BD Biosciences, San Jose, Calif.), CD8β-PC5, T-cell receptor αβ-PC5 (Beckman Coulter, Brea, Calif.), CD127-APC (eBioscience, San Diego, Calif.) and analyzed on the Gallios Flow Cytometer.

### Data Analysis

For determination of the relative number of NSG repopulating units (NRU) in CD34+ or CD34+/CD90+ cells, generalized linear models were used to estimate the parameters from a single-hit Poisson model Li M J and Rossi J J. Lentiviral vector delivery of recombinant small interfering RNA expression cassettes. Methods in enzymology. 2005; 392:218-226; Harden J. and Hilbe J. Generalized Linear Models and Extensions: Stata Press 2011. The analyses were done using the glm package in the R statistical framework. The data for this engraftment assay included 182 mice that received one of 12 doses of CD34+ cells from 4 donors with CD90 percentages ranging from 50-80%. Engraftment was defined as a value of natural log (ln) CD45% in bone marrow >1. Cell treatment (minimally cultured (MC-HSPC), expanded (EXP-HSPC)) and prior cryopreservation (yes, no) were included in the models as explanatory variables. The slope coefficient for ln (dose) for CD34+ cells was 1.04 (standard error=0.20; z=0.2, two-tailed p-value>0.5) and the slope coefficient for ln (dose) for CD34+/CD90+ cells was 0.92 (standard error=0.13; z=−0.62, two-tailed p-value>0.5) indicating the single-hit Poisson model is appropriate for both cell dose models. The predicted active cell frequency (one NSG repopulating unit) is the reciprocal of the exponent of the intercept terms based on cell treatment and prior cryopreservation in the single-hit Poisson model with the slope for ln (dose) set to 1. The area under the responder operator curve for the CD34+ and CD34+/CD90+ models was 0.90 and 0.89, respectively indicating that the models correctly classified the engraftment 90% and 89% of the time. For all other analyses, either analysis of variance or a two-sample two-tailed Student's t-test was employed. The level of significance was set to 0.05.

Bone marrow of animals transplanted with either fresh or expanded adult HSPC contained secondary engrafting cells at 16 weeks, verifying the presence of primitive stem cells in both populations. HSPC genetically modified with a lentiviral vector encoding a drug resistance gene (MGMT^{p140k}) were also used to reconstitute NSG mice. In vivo treatment with 2-3 rounds of bis-chloronitrosourea (BCNU) with O⁶-BG resulted in a 4-8 fold enrichment in GM progeny in both the bone marrow and spleen. Thus herein, methods have been developed for increasing the number of adult, GM HSPC for transplantation and enriching for GM cells in vivo following engraftment. Additionally, the adult NSG mouse model was qualified as a useful tool for quantification of the effects of ex-vivo manipulations of HSPC intended for clinical use.

### Example 2

### Durable Multi-Lineage Engraftment in Adult NSG Mice Transplanted with Adult HSPC

### Mouse Transplantation

Adult (8-10 week old) NSG mice were irradiated at 270 cGy 24 hours prior to transplantation. Mice were injected (IV) with doses ranging from 0 to 2×10⁶ CD34+ HSPC per animal (fresh) or 0 to 4×10⁶ CD34+ HSPC (expanded) in saline for injection (APP Pharmaceuticals, Lake Zurich, Ill.). Mice were transplanted with fresh CD34+ HSPC cells, after overnight stimulation as described above. Some animals received 20 μg Fc/IL-7 protein intravenously for up to 16 weeks as indicated. For secondary engraftment experiments, primary animals were transplanted with 7.44×10⁵ fresh or 2.1×10⁶ expanded CD34+ cells and maintained for 16 weeks as above. At 16 weeks post-transplant, bone marrow cells from primary mice that received fresh HSPC were harvested, pooled and used to transplant 8 secondary recipient mice with 3.0×10⁷ total bone marrow cells per mouse containing 2.1×10⁶ CD34+/CD45+ cells. Similarly, the bone marrow from mice transplanted with expanded HSPC was pooled and used to transplant 6 secondary recipient mice with 3.7×10⁷ total bone marrow cells per mouse containing 7.4×10⁵ CD34+/CD45+ cells. Bone marrow from both sets of secondary recipients was analyzed 16 weeks after transplant for human cell engraftment.

### Lentiviral Vector Preparation and HSPC Transduction

Lentiviral vector was produced by calcium phosphate precipitation in HEK293T cells as previously described by Li M J et al. (2005). CD34+ cells were pre-stimulated overnight in SFT6. Lentiviral transduction was conducted at 1×10⁶ cells/mL on Retronectin™ (Takara, Otsu-Shiga Japan)-coated non-tissue culture T-75 flask for 24 h in the presence of 20 ng/μL rapamycin (Sigma-Aldrich, St. Louis, Mo.). Lentivirus and rapamycin were removed and cells were washed once before being resuspended for transplantation in 0.9% NaCl saline solution with Fc/IL-7 as described above.

### Flow Cytometric Analysis of Engraftment

Mice were necropsied at 16 or 25 weeks post transplantation for analysis of engraftment. Single cell suspensions of bone marrow (femurs) and spleen were prepared by mechanical dissociation and red cells lysed using ACK lysis Buffer (Sigma-Aldrich, St. Louis, Mo.). All cell suspensions were pre-treated with human immunoglobulin (GammaGard, Baxter Healthcare Corp. Deerfield, Ill.) for 30 minutes to block nonspecific antibody staining. Cell suspensions were stained with a human pan-leukocyte antibody to CD45-PC5 (BioLegendR, San Diego, Calif.), and lineage specific antihuman, CD3-ECD, CD4-APC, CD14-APC-Alexa-750, CD19-PE (Invitrogen, Carlsbad, Calif.), and CD8β-PC5 (Beckman Coulter, Brea, Calif.), for 20 minutes and washed 2 times with 1 mL of PBS containing 0.1% BSA (Sigma-Aldrich, St Louis, Mo.). BM cells were stained with antibodies to human CD45-PC5, CD34-PC7, CD45-ECD (Beckman Coulter, Brea, Calif.), CD33-PC5 (BD Biosciences, San Diego, Calif.), CD19-PE, and CD14-APC-Alexa750 (Invitrogen, Carlsbad, Calif.) for 20 minutes and washed 2 times with 1 mL of PBS containing 0.1% BSA. To establish analytical gates and background staining, bone marrow and spleen samples from 2-3 mice that received no human cells mice were stained with the same antibody panel. Samples were analyzed using a Gallios flow cytometer (Beckman Coulter, Hialeah, Fla.) and data analyzed with FCS Express software (De Novo Software, Los Angeles, Calif.).

### Flow Cytometric Analysis of HSPC Expanded In Vitro

A representative aliquot of each expanded HSPC population was analyzed for stem cell content at day 7 of expansion. Cells were counted by Guava Via Count (Millipore, Billerica, Mass.), and approximately 2.5×10⁵ cells were stained with antibodies to CD34-PC7 (Beckman Coulter, Brea, Calif.), CD90-APC, and CD49f-PE (BD Biosciences, San Diego, Calif.), for 20 minutes on ice and washed 3 times with PBS containing 0.1% BSA (Sigma-Aldrich, St Louis, Mo.). 0.5 μg/mL DAPI (Invitrogen, Carlsbad, Calif.) was added as a live/dead cell discriminator. For isotype controls, matching Ig isotypes and conjugation from the same vendors were used. Samples were analyzed using Gallios Cytometer (Beckman Coulter, Brea, Calif.) and data analyzed with FCS Express software (DeNovo Software, Los Angeles, Calif.).

In human stem cell transplantation, CD34 dose is the one of the best predictors of engraftment and >2×10⁶ CD34+ cells/kg is the recommended dose to ensuring hematopoietic recovery following marrow-ablative conditioning. The CD90+(Thy-1+) sub-population of CD34+ cells contains more primitive HSPC and accounts for most of the long term engraftment in animal transplant models. In order to perform quantitative analysis of engraftment before and after stem cell expansion, dose-response curves were developed for engraftment of minimally manipulated adult HSPC in NSG mice based on CD34+ and CD34+/CD90+ cell dose. CD34+HSPC were isolated from G-CSF mobilized peripheral blood from 5 independent healthy donors by magnetic bead selection and cultured overnight in SFT6 (hereinafter referred to as minimally cultured or MC-HSPC) prior to transplant. Animals with doses of 0.5×10⁶, 1×10⁶ or 2×10⁶ CD34+MC-HSPC per mouse to establish the relationship between HSPC dose and engraftment were transplanted. The frequency of CD90+ cells, while not used prospectively in dosing, was recorded for each transplant cohort and the data used in subsequent statistical analysis of engraftment. Phenotypic analysis of blood, bone marrow, and spleen was performed using antibody to the human pan-leukocyte maker CD45 to identify human cells and calculate extent of engraftment. There was no evidence for human cells in the thymus or lymph nodes of these animals.

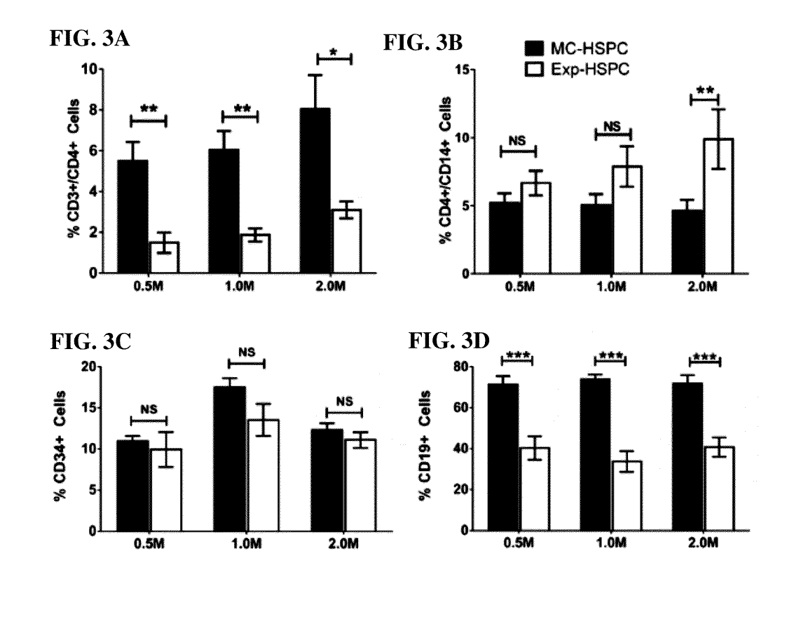

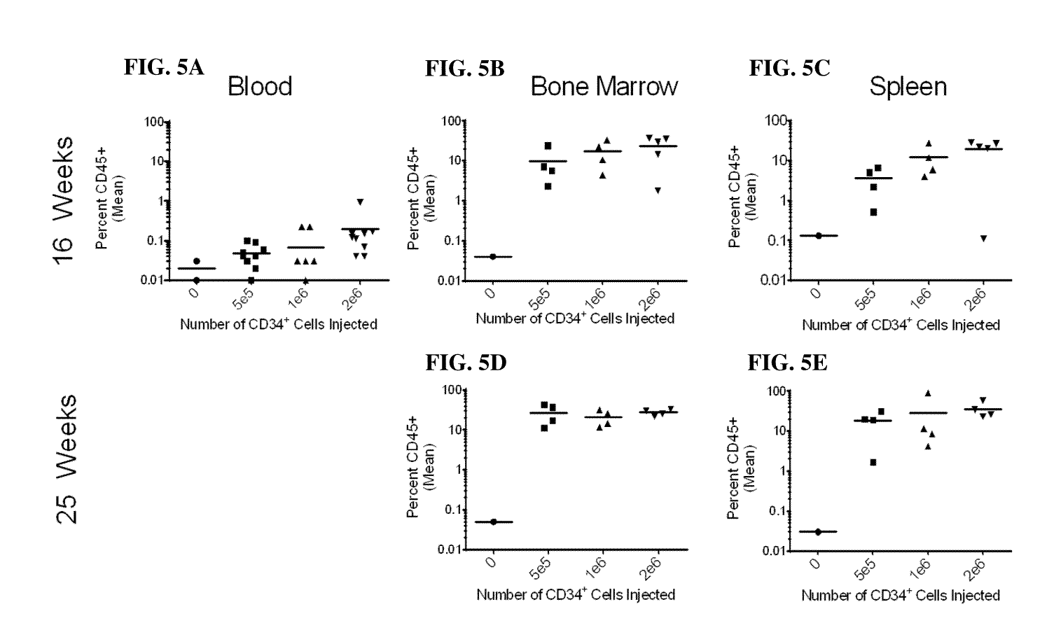

In contrast to previous reports in which cord blood progenitors were used to engraft neonatal NSG mice [45-50], very low levels were detected (<0.5%) of human cells in the peripheral blood of most mice at 16 weeks after transplant (FIG. 5A). However, the average human cell engraftment in animals receiving at least 0.5×10⁶ CD34+MC-HSPC was 9.8% in bone marrow (FIG. 5B) and 3.8% in spleen (FIG. 5C). Increasing the dose to 1 or 2×10⁶ CD34+MC-HSPC resulted in modest increases in the level of CD45+ cell engraftment in the bone marrow (18±6.4% and 24±6.8% respectively) and spleen (12±5.2% and 19±5.1% respectively) but these were not significantly different from the 0.5×10⁶ dose. Peripheral blood engraftment was not followed subsequently, as it was not predictive of organ engraftment. Animals from the same cohort were maintained for 25 weeks after transplant and then analyzed for engraftment. All animals were engrafted at levels comparable to or greater than that seen at 16 weeks in this cohort demonstrating the durability of engraftment with this source of cells (FIGS. 5D-5E).

### Example 3

### Analysis of Bone Marrow and Spleen of Mice at 16 Weeks after Transplantation for Distribution of Lymphoid, Myeloid and Hematopoietic Progenitor Cells

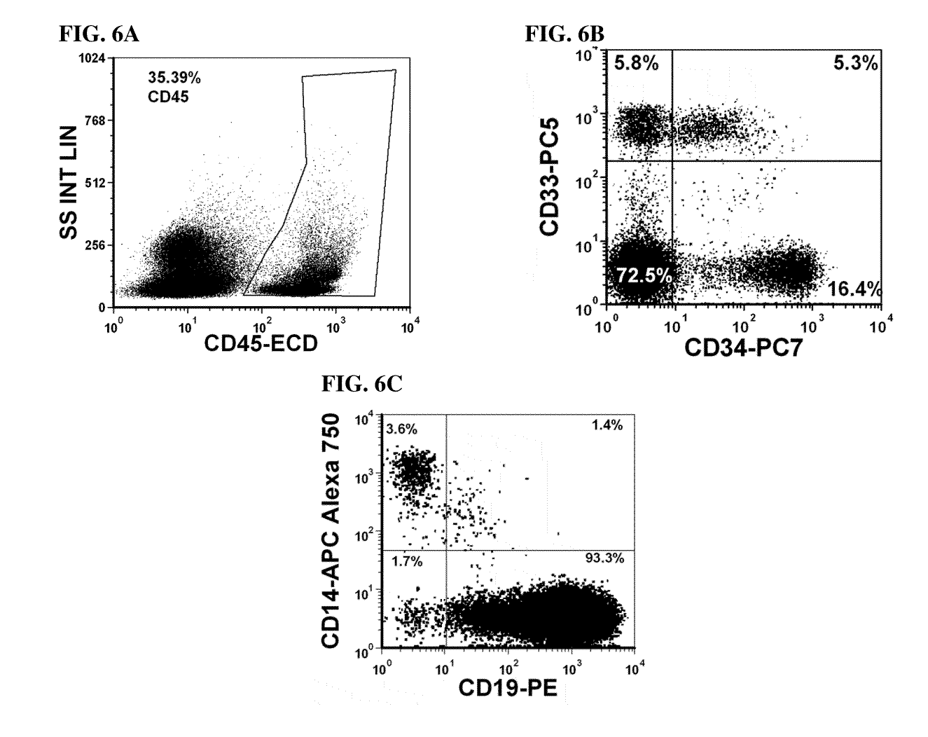

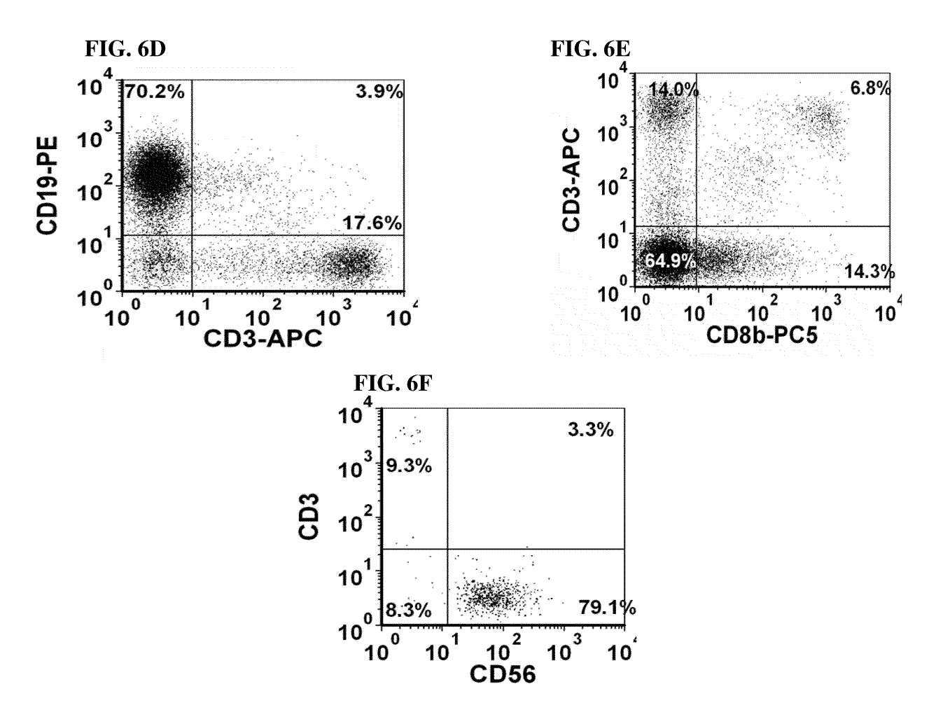

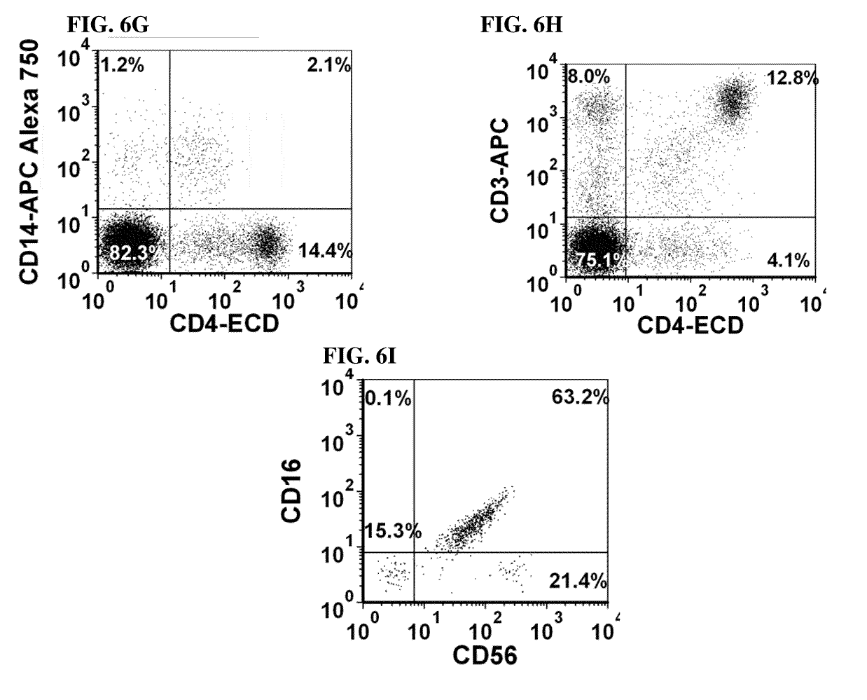

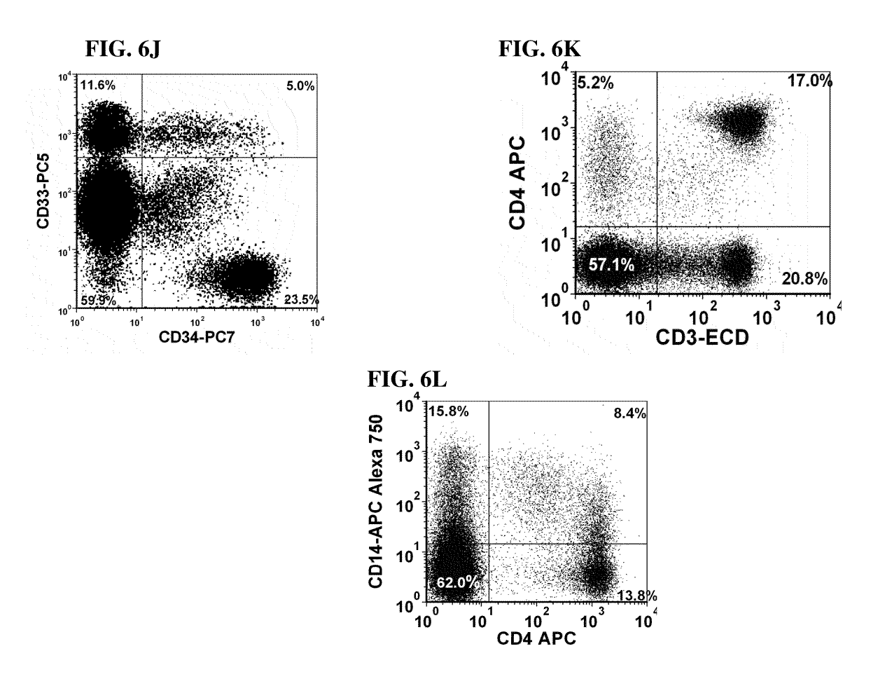

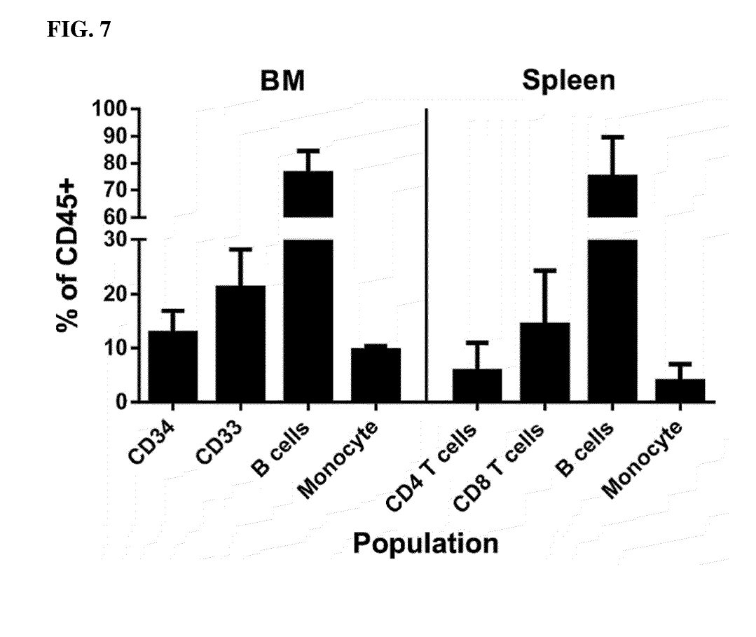

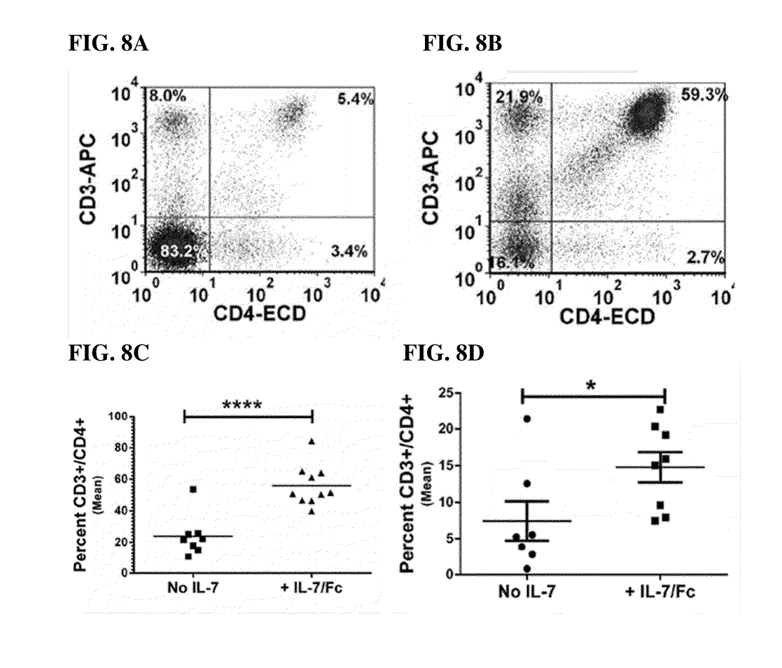

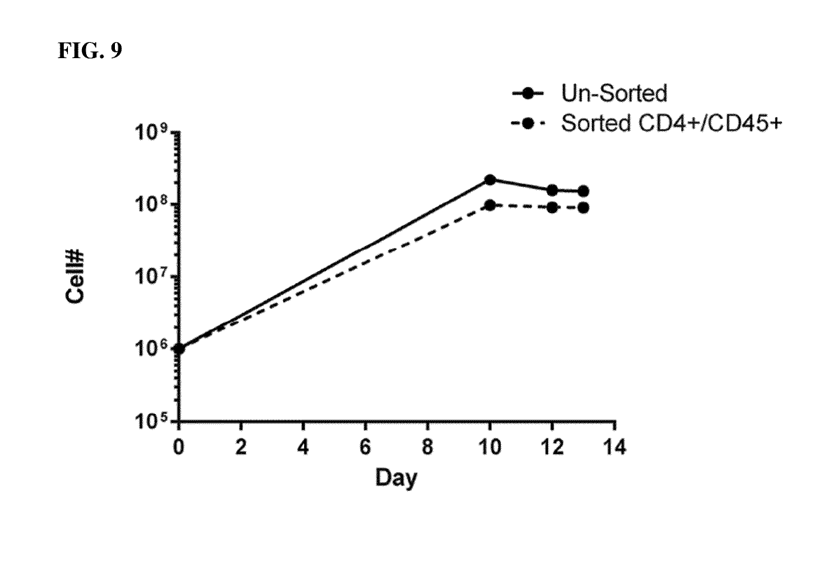

The bone marrow and spleen of mice at 16 weeks after transplantation for distribution of lymphoid, myeloid and hematopoietic progenitor cells were analyzed. The marrow of engrafted mice contained predominantly CD19+ B-cells with evidence of CD4+/CD14+ monocytes as wells as CD34+(multipotent) and CD33+(myeloid) progenitors (FIGS. 6A-6C). The spleens also contained CD4+ and CD8+ T-cells, CD19+ B-cells, CD4+/CD14+ monocytes and CD16+/CD56+ NK Cells (FIGS. 6D-6I). Similar analysis of engraftment was performed at 25 weeks after transplantation and the grafts were found to be durable, containing multiple lymphoid and myeloid lineages and CD34+ cells (FIGS. 6J-6L). The distribution of lineages observed in >200 animals from cohorts transplanted with MC-HSPC from 5 independent donors was similar to that observed in the initial cohort described above. Taken together, this data demonstrates that transplantation of as few as 500,000 adult CD34+ HSPC leads to long term engraftment of multiple lymphoid and myeloid lineages in the spleen and bone marrow and that transplant of neonatal mice is not required for the development of T-cells.

### Example 4

### Extent of HSPC from Adult Growth Factor Mobilized Peripheral Blood Expansion In Vitro

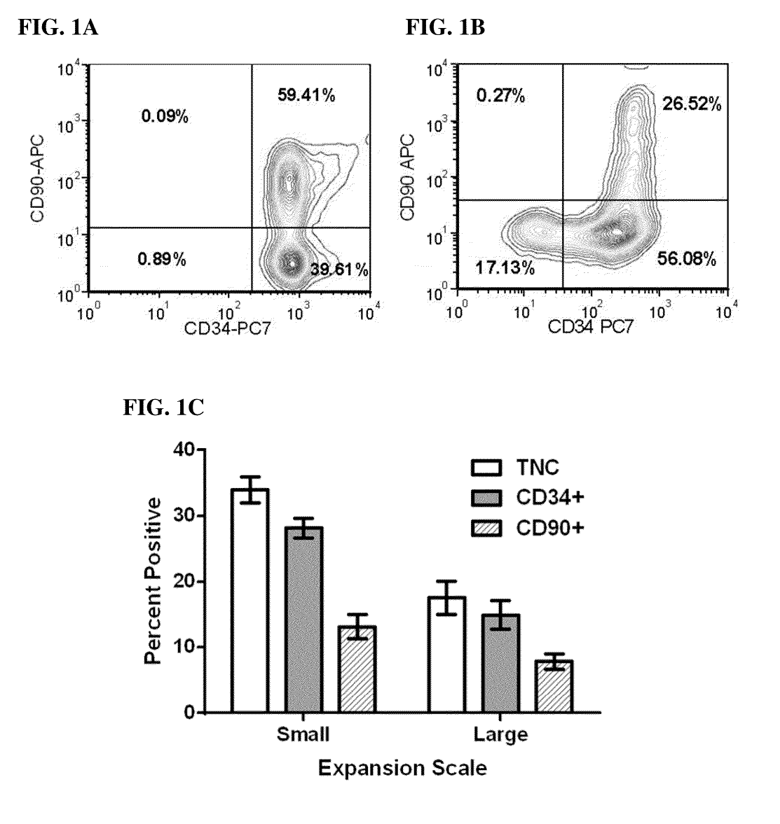

In order to establish the extent to which HSPC from adult growth factor mobilized peripheral blood could be expanded in vitro, known numbers of CD34+ HSPC from 5 healthy donors were cultured overnight in SFT6 (MC-HSPC) or for 7 days in SFT6 plus 0.75 μM SR-1 (Exp-HSPC) to assess the expansion of cells with a CD34+ HSPC phenotype. The CD90+(Thy-1+) sub-population of CD34+ cells contains more primitive HSPC and accounts for most of the long term engraftment in animal transplant models. Therefore, all cultures were analyzed for total nucleated cells count (TNC) and the frequency of CD34+ and the more primitive CD34+/CD90+ cells before and after culture. The starting population of MC-HSPC were >98% CD34+ and 24-82% of the CD34+ were also CD90+ as shown in a representative example in FIG. 1A. The frequency of CD34+ and CD34+/CD90+ cells did not change appreciably after overnight pre-stimulation in SFT6. However, after one week of expansion culture, a marked reduction in the frequency of the more primitive CD34+/CD90+ Exp-HSPC (Range=15-41%) was observed (FIG. 1B). Nonetheless, an overall expansion of TNC (23.0±3.4-fold), CD34+ cells of (18.6±3.1-fold) and CD34+/CD90+ cells (9.5±0.8-fold) was observed (FIG. 1C).

### Example 5

### Hematopoietic Potential of Ex Vivo Expanded HSPC

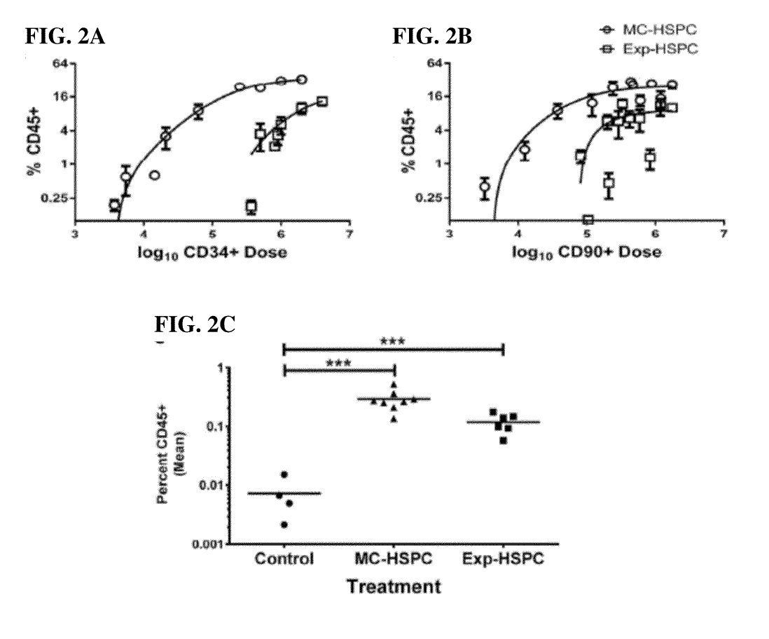

In order to establish the hematopoietic potential of in ex-vivo expanded HSPC, >280 NSG mice with varying doses of MC-HSPC or Exp-HSPC from 5 healthy donors were transplanted. CD34+ and CD34+/CD90+ cell dose infused were recorded for each animal. A significant increase in the number of Exp-HSPC required for engraftment compared to the MC-HSPC in both phenotypes was noted (FIGS. 2A-2B). The absolute level of engraftment of human cells in the mice (% CD45+) reached an average of 13.2±2.1% at the highest dose of Exp-HSPC tested (4×10⁶ CD34+ cells) while MC-HSPC reached 33.6±3.4% at the highest dose tested (2×10⁶ CD34+ cells). As both multipotent progenitors and stem cells are capable of engrafting primary recipient mice, secondary transplants of bone marrow from primary mice receiving either MC-HSPC or Exp-HSPC were performed. Femurs from mice receiving HSPC were pooled and the CD34+ HSPC content and total nucleated cell count was determined. For MC-HSPC, 8 mice received 3.0×10⁷ total bone marrow cells per mouse containing 2.1×10⁶ CD34+/CD45+ cells. For Exp-HSPC, 6 secondary recipient mice with 3.7×10⁷ total bone marrow cells per mouse containing 7.4×10⁵ CD34+/CD45+ cells. Each cohort was analyzed for the presence of human cells 16 weeks after secondary transplant (FIG. 2C). Animals receiving secondary transplants of bone marrow from both MC-HSPC (N=8) and Exp-HSCP (N=6) engrafted primary mice were engrafted with human cells at low but significant levels above the background of mice receiving only saline (p≦0.001). Therefore, primitive hematopoietic stem cells were present in each population used to transplant the primary mice.

In order to further characterize the stem cell content of MC-HSPC and Exp-HSPC, bone marrow from primary animals transplanted with each population was used to engraft secondary recipients. Bone marrow from each cohort was analyzed for the presence of human cells 16 weeks after secondary transplant. The results indicate that both MC-HSPC and Exp-HSPC were capable of engrafting secondary recipients (FIG. 2B) although too few HSPC were obtained to perform limiting dilution analysis during secondary transplant.

### Example 6

### Frequency of an Engrafting Dose in Minimally Cultured and Expanded Populations

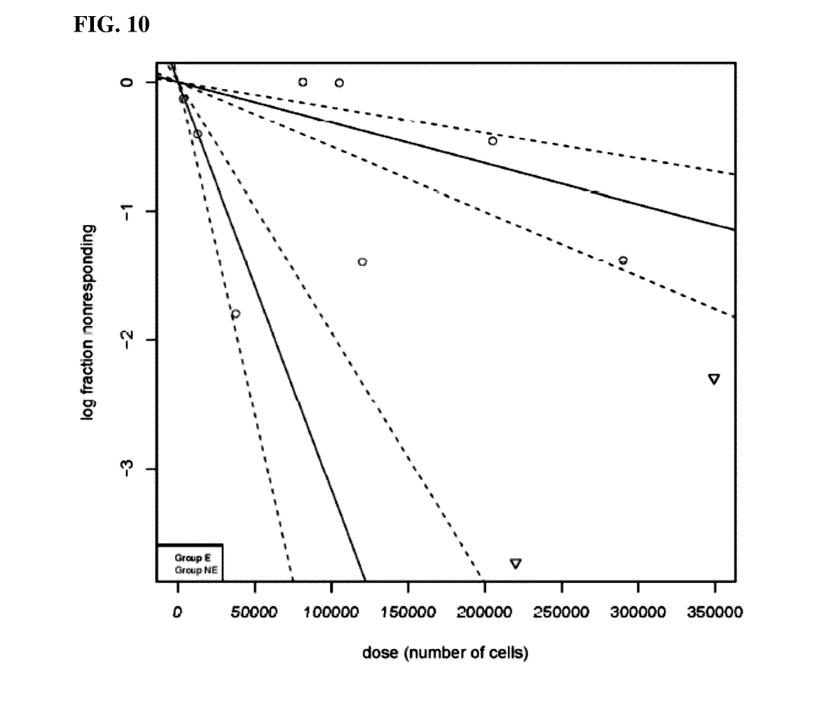

In order to estimate the frequency of an engrafting dose in minimally cultured and expanded populations, extreme limiting dilution analysis of 68 mice transplanted with MC-HSPC and 38 mice receiving Exp-HSPC was performed. CD34+/CD90+ dose to estimate the frequency of NSG engrafting activity in the two populations were used. Animals were transplanted with limiting doses of CD34+/CD90+ HSPC ranging from 3250-220000 cells (MC-HSPC) and 81250-349300 cells (Exp-HSPC) which included engraftment rates from 0% to 100% for each of the groups but did not include doses above the minimum dose that resulted in 100% engraftment. Engraftment was defined as ≧1% CD45+ cells in bone marrow at 16 weeks. The slope coefficient for CD34+/CD90+ cells (0.94) was not different from 1, indicating the single-hit Poisson model is appropriate. The estimated frequency of NSG-repopulating units (NRU) in the CD34+/CD90+ population was 10 fold lower (Range 5.11-19.67) in the Exp-HSPC compared to the MC-HSPC (FIG. 10). When considering that the mean expansion of CD34+/CD90+ cells was 9.5±0.8 during 7 days of culture in SFT6+SR-1, no expansion of NRU in adult HSPC during culture in SFT6+SR-1 was concluded.

### Example 7

### Expanded Adult HSPC have Reduced Lymphoid Potential that can be Partially Restored Through Administration of Fc/IL-7

### Fc/IL-7 Production

Fc/IL-7 was cloned into an OptiVect-TOPO™ (Invitrogen, Carlsbad, Calif.) vector and protein was produced from a cloned transfected DG44 CHO cell line as per the methods of Lo et al. High level expression and secretion of Fc-X fusion proteins in mammalian cells. Protein Engineering. 1998, 11: 495-500.

Expanded adult HSPC have reduced lymphoid potential that can be partially restored through the administration of human Fc/IL-7. Phenotypic analysis of the bone marrow and spleen of >200 primary animals transplanted were performed with MC-HSPC or Exp-HSPC. Both populations of HSPC engrafted and produced multiple lymphoid and myeloid lineages but the frequency of CD4+ T-cells and CD19+ B-cells among the human CD45+ cells in the spleen of animals receiving Exp-HSPC was significantly reduced compared to animals transplanted with MC-HSPC (P<0.05) and monocytes were significantly increased at the highest Exp-HSPC dose tested (p<0.01) (FIGS. 3A-3D). No significant differences were observed in the level of CD3+/CD8+ T-cells in the spleen or CD34+ cells in the bone marrow of animals at 16 weeks after transplantation with MC-HSPC and Exp-HSPC. These results demonstrated that in addition to the reduction in the frequency of in vivo engrafting cells among the Exp-HSPC population, there was an intrinsic deficit in CD4 T-cell and B-cell potential. There was no tumors in any mice and >90% of mice survived to the endpoint of 16 or 25 weeks in these assays.

The average level of CD4+ T-cells was low in all animals engrafted with either MC-HSPC or Exp-HSPC at 16 weeks (5-7% of CD45+ cells in the spleen) and did not increase significantly with CD34+ HSPC dose. To explore HIV infection in these mice, a human Fc/IL-7 fusion protein to enhance T-cell engraftment as has been previously described (Mezquita P, et al. NOD/SCID repopulating cells contribute only to short term repopulation in the baboon. Gene therapy. 2008; 15:1460-1462; Chung J, et al. Combinatorial RNA-based gene therapy for the treatment of HIV/AIDS. Expert opinion on biological therapy. 2013; 13:437-445) was cloned, expressed and purified. Cohorts of animals transplanted with MC-HSPC and treated with Fc/IL-7 showed a significant (2.3-fold, P<0.0001) increase in the average level of CD3+/CD4+ splenic T-cells while Exp-HSPC also showed a significant (2-fold, P<0.05) increase in the average level of CD3+/CD4+ T-cells in the spleen of mice 16 weeks after transplantation. CD8+ T-cells, although not reduced in Exp-HSPC transplanted mice, also increased as a result of Fc/IL-7 treatment. The fraction of CD45+ cells that were CD19+ B-cells was reciprocally reduced as a result of the expansion of T-cells in Fc/IL-7 treated animals. This demonstrates that lymphopoiesis in adult HSPC can be augmented with exogenous human IL-7 treatment.

### Example 8

### Characterization of T-Cells from Spleens of Humanized Mice

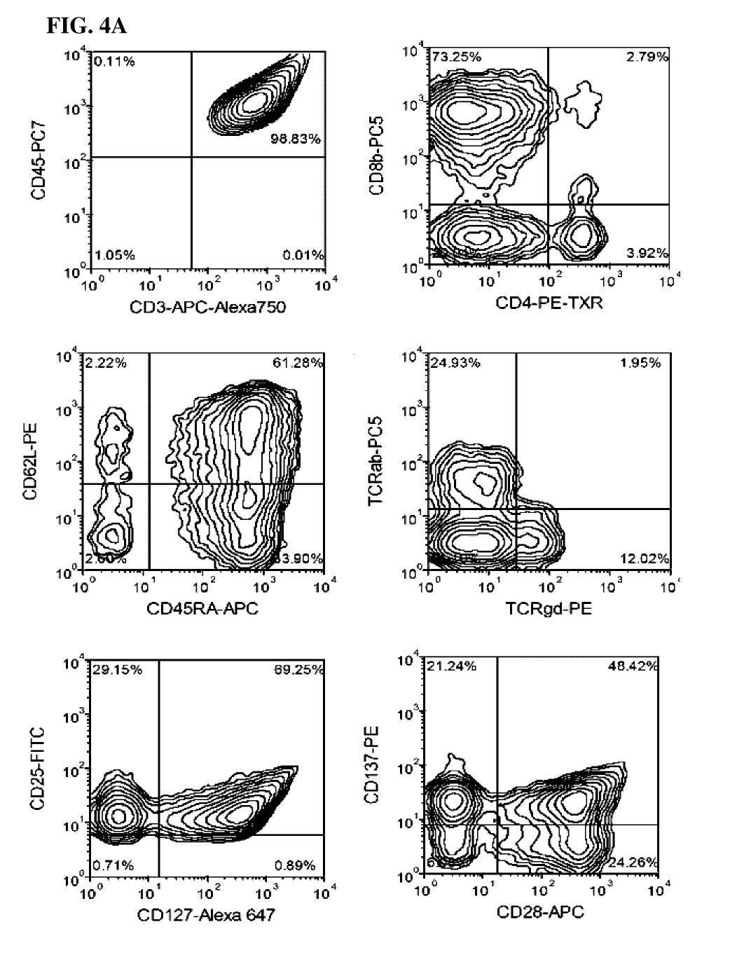

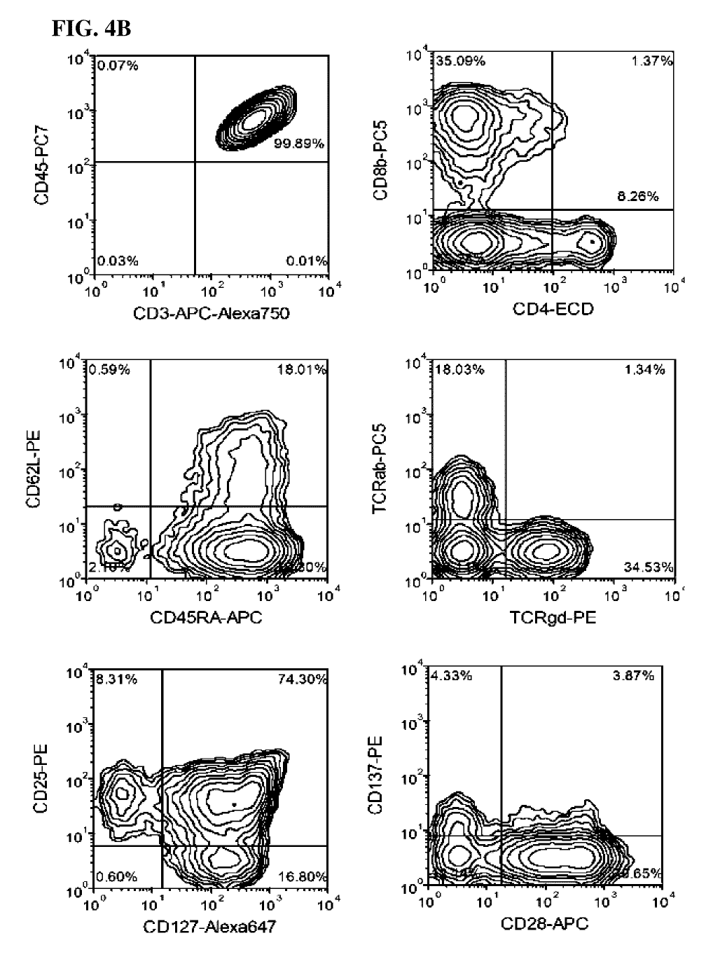

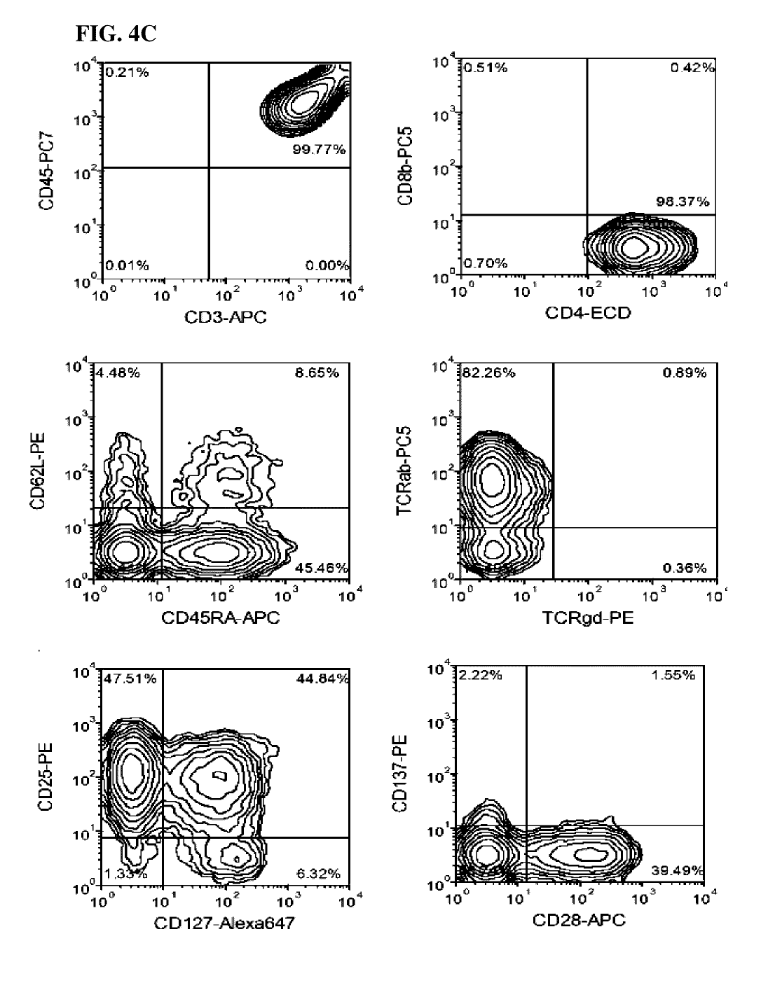

Whole spleen and purified CD4+ T-cells were isolated from transplanted mice and the phenotype and proliferative potential of the isolated cells were characterized. Whole splenocytes and sorted CD4+ T-cells were activated and cultured with irradiated allogeneic feeder cells for two weeks in the presence of IL-2 and IL-15. Expansion of CD3+ T-cells from splenocytes and sorted CD4+ cultures of was 153 and 91 fold respectively during the second round of culture. Cells from the first REM culture were stained with a cocktail of antibodies to lineage, homing receptors, cytokine receptors, antigen receptors and co-stimulatory molecules. After the first 12 days of culture of whole spleen, virtually all cells were human (CD45+) and expressed CD3 (T-cells) (FIG. 4A) The majority of the cells were CD3+/CD8+/CD4− but a small fraction (4%) were CD3+/CD4+/CD8− mature T-cells. The majority of cells were CD45RA+(naive) T-cells of which most (66%) expressed the CD62L (L-selectin) lymph node homing receptor. A fraction of cells (25%) expressed the αβ T-cell antigen receptor while approximately 12% expressed the γδ T-cell receptor. Almost all of the T-cells expressed the high affinity IL-2 receptor (CD25) with about ⅔ also expressing the IL-7 receptor (CD127). A large proportion of cells also expressed T-cell co-stimulatory ligands CD137 (4-1BB) and CD28. Thus, the cells grown out in the first round of rapid expansion method (REM) appeared to be largely naive T-cells that expressed antigen specific receptors, could potentially home to lymph nodes, and were also expressing co-stimulatory and cytokine receptors, indicating their potential to receive additional growth and proliferation signals.

After a second round of culture of whole spleen, an increase in CD3+/CD4+ cell content was observed but the percentage of cells expressing CD62L dropped and the percentage of cells expressing the γδTcR was increased approximately 3-fold (FIG. 4B). Most cells expressed IL-2 and IL-7 receptors although some cells (16%) down-regulated IL-7 receptor expression to background levels. Also noteworthy was the almost complete loss of the CD137 (4-1BB) co-stimulatory receptor during the second culture period. CD4+ T-cells isolated from the same spleen showed similar patterns of phenotype although CD3+/CD8+ and γδ T-cells were largely absent in these cultures and more cells expressed the high affinity IL-2 receptor only (FIG. 4C).

### Example 9

### Gene Modified HSPC can be Selectively Enriched in In Vivo Using O⁶BG and BCNU

Lentiviral vectors expressing unique combinations of small anti-HIV RNAs expressed from a naturally occurring micro RNA cluster (MCM-7) are known. See Chung J, et al. Endogenous MCMI microRNA cluster as a novel platform to multiplex small interfering and nucleolar RNAs for combinational HIV-1 gene therapy. Human gene therapy. 2012; 23:1200-1208. The mutant (MGMT^{p140k}) gene was subcloned into these constructs in order to assess the ability to selectively enrich for GM-HSPC and their progeny in vivo. CD34+ HSPC were transduced with this vector and used to transplant NSG mice as described in materials and methods. Animals were treated with O⁶BG and BCNU at weeks 7 and 8 (2× treatment) or weeks 7, 8 and 9 (3× treatment) following transplantation. Two weeks later (11 weeks following transplantation) animals were necropsied and the level of engraftment of the spleen and bone marrow were determined.

The results demonstrated that engraftment of the spleen and bone marrow with human cells was reduced in both 2× and 3× treated animals relative to the untreated control (p<0.0001) but the frequency of CD45+/GFP+ cells in the spleen and bone marrow was enriched 10 and 15-fold in the 2× and 3× treated cohorts, respectively. The average frequency of CD3+/CD4+ T-cells in the spleen increased 3-fold when O⁶BG/BCNU treatment was performed three times. Similarly, CD4+/CD14+/GFP+ monocytes were increased 3-fold in the spleens of mice following three O⁶BG/BCNU treatments. No deficiencies in lineage development were noted in the drug treated mice.

In a second experiment performed with a different donor HSPC product, the cells were allowed to recover from O⁶BG/BCNU treatment for 7 weeks after the last drug treatment (with weekly Fc/IL-7 treatment) before harvesting spleens for assessment of engraftment and GFP+ cell content. Similar to the first experiment, depletion of CD45+ cells was observed in the bone marrow and spleen with each O⁶BG/BCNU treatment and a concomitant increase in the frequency of CD45+/GFP+ human cells and CD14+/CD4+/GFP+ monocytes but no significant enrichment of CD3+/CD4+/GFP+ T-cells. There was enhanced recovery of the T-cell compartment from non-modified stem cells remaining in the bone marrow and was mediated by the Fc/IL-7 treatments.

### Example 10

### Immunodeficient (NSG) Mouse Model to Quantitatively and Qualitatively Assess In Vitro Expansion of Adult HSPC and their Progeny

Dose response curves for engraftment were established using limiting numbers of MC-HSPC as a basis for quantitative comparison of MC-HSPC with Exp-HSPC. Phenotypic analysis of populations before and after expansion combined with quantitative analysis of in vivo engraftment established the relationship between phenotype and function of HSPC. Secondary engraftment and lineage analysis were used to verify the presence of primitive stem cells in each population and assess any defects in multi-lineage hematopoietic potential, respectively.

Investigation of lineage development in transplanted animals revealed that engraftment of both MC-HSPC and Exp-HSPC was multi-lineage and included both CD3+/CD4+ T-cells and CD4+/CD14+ monocytes which are targets for HIV infection and thus critical for evaluating the HIV gene therapy strategy in vivo. Engraftment of both populations was also durable, lasting for at least 25 weeks after transplant. Cells capable of engraftment of secondary recipients were present in the bone marrow of the primary recipients for at least 16 weeks, which confirmed the presence of primitive hematopoietic stem cells in the primary grafts. No human cell engraftment of lymph nodes or thymus and minimal engraftment of cells in the circulation of these mice was observed. This is in contrast to observations in neonatal animals transplanted with umbilical cord blood or fetal liver-derived HSPC.

Using a single hit kinetic model of engraftment, it was determined that the frequency of NSG engrafting cells was reduced approximately 10-fold in the Exp-HSPC relative to MCHSPC. The relative expansion of CD34+/CD90+ cells in vitro during 7-day cultures was also approximately 10-fold, indicating that in vivo engrafting units were maintained but not expanded under these conditions. There was a significant discordance between in vitro estimates of expansion by stem cell surface phenotype and in vivo estimates of engraftment. Even the primitive stem cell phenotype (CD34+/CD90+) did not maintain fidelity with biological activity. The skewing of engraftment away from the lymphoid lineage and towards myeloid lineage was also noteworthy and similar to results from prior HSPC expansion studies.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A humanized mouse comprising a gene-modified human hematopoietic stem and progenitor cell (GM-HSPC) graft, wherein HSPC cells within said graft are transduced with a gene vector, wherein said gene vector comprises a drug resistance gene or a disease treatment gene.

2. The humanized mouse of claim 1, wherein said gene vector comprises drug resistance gene.

3. The humanized mouse of claim 2, wherein said drug resistance gene is a bis-chloronitrosourea (BCNU) resistance gene.

4. The humanized mouse of claim 1, wherein said gene vector comprises a disease treatment gene.

5. The humanized mouse of claim 4, wherein said disease treatment gene is a HIV disease treatment gene.

6. A humanized mouse comprising an adult human hematopoietic stem and progenitor cell (HSPC) graft, wherein the graft is formed by a method comprising:
i) contacting a plurality of isolated adult human HSPCs with a cytokine culture media comprising stem cell factor (SCF), FMS-like tyrosine kinase-3 ligand (Flt-3L), thrombopoietin (TPO), and interleukin-6 (IL-6), thereby forming a plurality of cultured-HSPCs;
ii) allowing said plurality of cultured-HSPCs to undergo expansion, thereby forming a plurality of CD34+ HSPCs;
iii) administering to a mouse, at least about 5×10⁵ of said plurality of CD34+ HSPCs; and
iv) allowing said CD34+ HSPCs to engraft in said mouse, thereby forming said humanized mouse.

7. The humanized mouse of claim 1, wherein said method further comprises contacting cultured HSPCs at step (ii) with an aryl hydrocarbon receptor (AhR) antagonist.

8. The humanized mouse of claim 7, wherein said aryl hydrocarbon antagonist is SR-1.

9. The humanized mouse of claim 1, wherein about 5×10⁵ to about 2×10⁶ of said plurality of CD34+ HSPCs are administered to said mouse.

10. The humanized mouse of claim 1, wherein the adult HSPCs are gene vector transfected HSPCs.

11. The humanized mouse of claim 1, wherein said plurality of CD34+ HSPCs differentiate to lymphoid cells.

12. The humanized mouse of claim 11, wherein said lymphoid cells are CD3+/CD8+/CD4− T-cells, CD3+/CD4+/CD8− T-cells, or CD3+/CD4+ T-cells.

13. The humanized mouse of claim 11, wherein said lymphoid cells are CD19+ B-cells, CD4+/CD14+ monocytes, or CD16+/CD56+ NK cells.

14. The humanized mouse of claim 1, wherein the method further comprises administering Fc/IL-7 to said mouse.

15. The humanized mouse of claim 10, wherein said gene vector is a lentiviral gene vector.

16. The humanized mouse of claim 15, wherein said gene vector comprises a drug resistance gene or a disease treatment gene.

17. The humanized mouse of claim 16, wherein said gene vector comprises drug resistance gene.

18. The humanized mouse of claim 17, wherein said drug resistance gene is a bis-chloronitrosourea (BCNU) resistance gene.

19. The humanized mouse of claim 16, wherein said gene vector comprises a disease treatment gene.

20. The humanized mouse of claim 19, wherein said disease treatment gene is a HIV disease treatment gene.

21.-31. (canceled)
